(19) United States
(12) Patent Application Publication
(10) Pub. No.: US 2009/0142753 A1
(43) Pub. Date: Jun. 4, 2009

(54) Compositions and methods for detecting compounds to treat a neurological disorder

(75) Inventor: Jin Xu, Wellesley, MA (US)

Correspondence Address: EDWARDS ANGELL PALMER & DODGE LLP, P.O. BOX 55874, BOSTON, MA, 02205, US

(73) Assignee: Caritas St. Elizeth Medical Center of Boston, Inc., Boston, MA (US)

(21) Appl. No.: 11/887,016

(22) PCT Filed: Mar. 17, 2006
(86) PCT No.: PCT/US2006/010046

§ 371 Date: Dec. 15, 2008

Related U.S. Application Data
(60) Provisional application No. 60/665,287, filed on Mar. 25, 2005.

(51) Int. Cl. C12Q 1/68; C12Q 1/02; A61K 49/00; C12N 15/63

(52) U.S. Cl. 435/6; 435/29; 424/9.1; 800/3; 435/320.1

(57) ABSTRACT

The present invention generally provides compositions and methods that can be used to detect compounds that modulate the activity of at least one of the DJ-1, Parkin and Pink-1 genes.

### CROSS-REFERENCE TO RELATED APPLICATION

This application claims the benefit of the following U.S. Provisional Application No. 60/665,287, which is hereby incorporated by reference in its entirety.

### BACKGROUND OF THE INVENTION

Parkinson's disease is a common neurodegenerative disorder characterized by the selective loss of dopaminergic neurons in the substantia nigra and reduced dopamine signaling in the striatum. Symptoms of Parkinson's disease include resting tremor, muscular rigidity, slowness of movement, and postural instability. In addition, some patients develop depression and cognitive impairment. Most therapeutic approaches target dopamine metabolism pathway, but do not always prevent neurodegeneration. A number of genes have been genetically linked to Parkinson's disease pathogenesis. Certain of these genes regulate the oxidative stress response (α-synuclein, DJ-1), protein degradation (Parkin), and mitochondrial functions (PINK-1, a-synuclein, Parkin). Mutations in these genes have been associated with early-onset PD. For instance, triplication or point mutations in the α-synuclein gene are believed to contribute to autosomal dominant Parkinson's disease. Deletions and point mutations in Parkin, DJ-1 and PINK-1 are associated with autosomal recessive Parkinson's disease. The accumulation of α-synuclein may cause neurodegeneration in dopaminergic neurons both in vitro and in vivo. In contrast, Parkin, DJ-1 and Pink-1 may be neuroprotective.

### SUMMARY OF THE INVENTION

The present invention provides compositions and methods that can be used to detect compounds that modulate the activity of at least one of the DJ-1, Parkin and Pink-1 genes. In one embodiment, compounds that increase the activity of at least one of these genes, for example, by enhancing transcription are useful in the methods of the invention. The invention has a wide spectrum of useful applications including identifying compounds that can be used to prevent, treat or reduce symptoms associated with certain neurological disorders.

The invention thus provides, in one aspect, a screen to determine the therapeutic capacity of a known or candidate compound to increase activity of at least one of the DJ-1, Parkin and Pink-1 genes. Monitored gene activity is typically tested at the transcriptional level although post-transcriptional activities may be impact the testing indirectly (e.g., increased transcript and/or protein stability).

A particular screen according to the invention provides, in one embodiment, a method for detecting presence of a compound that can prevent, treat or help alleviate the symptoms of a neurological disorder. The method includes at least one of and preferably all of the following steps: (a) contacting a recombinant cell or cell line made in accord with the invention with at least one candidate compound; (b) incubating the cells under conditions sufficient to express a detectable reporter sequence; and (c) detecting a change in the expression of the reporter sequence (relative to a suitable control) as being indicative of the presence (or absence) of a compound that can modulate one or more of the DJ-1, Parkin and Pink-1 gene promoters. In other embodiments, the invention provides methods of identifying a compound that increases DJ-1, Parkin, or Pink-1 expression in a cell. The method involves (a) providing a cell expressing a Parkin promoter; (b) contacting the cell with a candidate compound; and (c) detecting Parkin expression, where an increase in the level of Parkin expression in the cell relative to a reference, identifies the candidate compound as a compound that increases Parkin expression.

In general, the invention provides a method of identifying a compound that increases DJ-1 gene expression in a cell. The method involves contacting a cell expressing a DJ-1 promoter (e.g., a heterologous promoter) with a candidate compound; and detecting an increase in DJ-1 gene expression, where an increase in the level of DJ-1 gene expression in the cell relative to a reference, identifies the candidate compound as increasing DJ-1 expression. In various embodiments, the DJ-1 promoter is present in an expression vector, or is operably linked to a detectable reporter and is detected by assaying the level of a detectable reporter. In yet another embodiment, the DJ-1 promoter is endogenously expressed in the cell and gene expression is detected by assaying mRNA level or by assaying protein level.

In another aspect, the invention provides a method of identifying a compound that increases Parkin gene expression in a cell. The method involves contacting a cell expressing a Parkin promoter (e.g., a heterologous promoter) with a candidate compound; and detecting Parkin gene expression, where an increase in the level of Parkin gene expression in the cell relative to a reference, identifies the candidate compound as a compound that increases Parkin gene expression. In one embodiment, the Parkin promoter is present in an expression vector. In another embodiment, the Parkin promoter is operably linked to a detectable reporter and Parkin gene expression is detected by assaying the level of a detectable reporter. In yet another embodiment, the Parkin promoter is endogenously expressed in the cell and gene expression is detected by assaying mRNA level, or by assaying protein level.

In yet another aspect, the invention provides a method of identifying a compound that increases Pink-1 gene expression in a cell. The method involves contacting a cell expressing a Pink-1 promoter (e.g., a heterologous promoter) with a candidate compound; and detecting Pink-1 gene expression, where an increase in the level of Pink-1 gene expression in the cell relative to a reference, identifies the candidate compound as a compound that increases Pink-1 gene expression. In one embodiment, the Pink-1 promoter is present in an expression vector. In another embodiment, the Pink-1 promoter is operably linked to a detectable reporter and Pink-1 gene expression is detected by assaying the level of the detectable reporter. In another embodiment, the Pink-1 promoter is endogenously expressed in the cell and Pink-1 expression is detected by assaying mRNA levelor by assaying protein level.

In yet another aspect, the invention provides a method for identifying a compound that modulates DJ-1, Parkin or Pink-1 gene expression. The method involves contacting a cell expressing a DJ-1, Parkin, or Pink-1 promoter operably linked to a detectable reporter with a candidate compound; and detecting a change in the expression of the detectable reporter relative to a control, thereby identifying a compound that modulates a DJ-1, Parkin or Pink-1 promoter.

In a related aspect, the invention provides a method for identifying a compound that treats or prevents a neurological disorder in a subject. The method involves contacting a cell comprising a DJ-1, Parkin, or Pink-1 promoter operably linked to a detectable reporter with a candidate compound; and detecting a change (e.g., an increase or a decrease) in the expression of the reporter sequence relative to a control, thereby identifying a compound that treats or prevents a neurological disorder. In one embodiment the reporter is detected by a fluorometric assay that involves the use of a fluorescence microscope, fluorometer, fluorescence microplate reader, fluorescence activated cell sorter or flow cytometer. In one embodiment, the assay signal is compared to a baseline signal produced by a control assay. In another embodiment, the baseline signal is subtracted from the assay signal to produce a corrected signal indicative of presence of the compound. In yet another embodiment, the method is in an automated, semi-automated, or manual format. In yet another embodiment, the method is a high throughput screening assay. In other embodiments, the assay involves the use of a computer interfaced with a fluorometer, fluorescence microplate reader, flow cytometer or luminometer to receive input from the assay and provide output data to a user where the output data is stored and optionally manipulated by the computer or outputted to the user in real-time.

In yet another aspect, the invention provides a expression vector comprising at least one of a DJ-1, Parkin or Pink-1 promoter sequence operably linked to at least one reporter sequence. In various embodiment, the DJ-1, Parkin or Pink-1 promoter sequence contains between 50 and 2000 base pairs upstream of the transcription start site, where the lower end of the range includes any integer between 49 and 1999, and the upper end of the range includes any integer between 51 and 2000 (e.g., 50, 100, 500, 1000, 1500, or 2000 base pairs upstream) of the DJ-1 Parkin or Pink-1 transcription start site. In one embodiment, the DJ-1, Parkin or Pink-1 promoter sequence of the vector is replaced with a sequence capable of hybridizing to the promoter sequence under high stringency conditions. In another embodiment, the DJ-1, Parkin or Pink-1 promoter sequence is replaced with a sequence that is at least 90% identical to a DJ-1, Parkin or Pink-1 promoter sequence. In yet another embodiment, the vector further comprises a DJ-1, Parkin or Pink-1 downstream sequence having between about 25 and 500 base pairs downstream from the human DJ-1, Parkin or Pink-1 transcription start site, where the lower end of the range includes any integer between 25 and 499, and the upper end of the range includes any integer between 26 and 500 (e.g., 25, 50, 100, 150, 200, 225, or 250 base pairs downstream from the human DJ-1, Parkin or Pink-1 transcription start site) in which each downstream sequence is operably linked to the promoter sequence. In one embodiment, the DJ-1, Parkin or Pink-1 downstream sequence is replaced with a nucleic acid sequence that is capable of hybridizing to a DJ-1, Parkin, or Pink-1 nucleic acid sequence. In another embodiment, the vector comprises a human DJ-1 promoter sequence comprising between from about 50 base pairs to 1000 base pairs upstream of the human DJ-1 transcription start site and is operably linked to a DJ-1 downstream sequence having about 200 base pairs downstream of the human DJ-1 transcription start site. In one embodiment, the DJ-1 downstream sequence is about 65 base pairs downstream of the human DJ-1 transcription start site. In another embodiment, the vector comprises a human Parkin promoter sequence comprising about 100 base pairs upstream of the human Parkin transcription start site, the promoter sequence further comprising an operably linked Parkin downstream sequence having about 50 base pairs to 200 base pairs downstream of the human Parkin transcription start site. In yet another embodiment, the Parkin downstream sequence is about 68 base pairs or less downstream of the human Parkin transcription start site. In yet another embodiment, the vector comprises a human Pink-1 promoter sequence comprising about 100 base pairs upstream of the human Pink-1 transcription start site, the promoter sequence further comprising linked in sequence a Pink-1 downstream sequence having about 10 base pairs to 200 base pairs downstream of the human Pink-1 transcription start site. In yet another embodiment, the Pink-1 downstream sequence is about 32 base pairs or less downstream of the Pink-1 transcription start site.

In yet another aspect, the invention provides an expression vector comprising a DJ-1, Pink1, or Parkin promoter operably linked to any one or more of the following: a polynucleotide encoding an ampicillin resistance gene or functional fragment thereof; an f1 origin sequence; an upstream synthetic poly(A) region; anyone of the promoter sequences described herein covalently linked to anyone of the downstream sequences; a polynucleotide sequence encoding a luciferase derivative; an SV40 late poly (A) signal; and a polynucleotide encoding a neomycin resistance gene; or functional fragment thereof.

In a related aspect, the invention provides a recombinant cell (e.g., a cell derived from neuronal, breast, testis or prostate tissue) comprising an expression vector of any previous aspect. In various embodiments, the recombinant cell is present in a cell line (e.g., a human 293, mouse NIH3T3, Chinese hamster ovary (CHO), HeLa or COS cell line). In another embodiment, the cell is derived from a neuroblastoma (e.g., a human neuroblastoma SH-SY5Y cell). In yet another embodiment, the cell is stably transformed by the expression vector.

In yet another aspect, the invention provides methods for producing a recombinant cell. The method involves contacting the cell with the expression vector under conditions conducive to introducing the vector into the cell; and transforming the cell to make a recombinant cell.

In yet another aspect, the invention provides a kit containing a recombinant cell of a previous aspect.

In yet another aspect, the invention provides a method of inhibiting neuronal cell death in a subject in need thereof. The method involves administering a compound identified in any previous aspect to the subject.

In a related aspect, the invention provides a method of treating a subject having a neurological disorder characterized by neuronal cell death, the method comprising administering a compound that increases the expression of at least one of DJ-1, Pink-1, or Parkin. In one embodiment, the method increases the expression of DJ-1. Desirably, the increase is by at least 5%, 10%, 25%, 50%, 75%, 100%, 200% or more.

In various embodiments of any previous aspect, the candidate compound (e.g., small molecule, protein, nucleic acid molecule, or fragments thereof) is a histone deacetylase inhibitor (HDAC). In other embodiments of any previous aspect, the candidate compound is a short-chain fatty acid, hydroxamic acid, cyclic tetrapeptide, or benzamide. In still other embodiments, the candidate compound is 4-phenylbutyrate, valproic acid, suberoylanilide hydroxamic acid (SAHA), pyroxamide, trochostatin A, oxamflatin, trapoxin A, apicidin, butyrate salt; or a derivative thereof. If desired, the screening method of a previous aspect further comprises testing the compound in an animal model (e.g., an animal that received before, during or after exposure to the candidate compound an amount of 1-methyl-4-phenyl-1,2,3,6-tetrahydropyridine (MPTP) or rotenone sufficient to cause symptoms associated with Parkinson's disease in the animal. In another embodiment, further testing of the screening method involves administering the compound to a transgenic animal (e.g., rodent) expressing α-synuclein. In various embodiments of any previous aspect, Parkinson's disease is assayed by detecting degeneration of a nigrostriatal pathway, raphe nuclei, locus ceruleus, or motor nucleus of vagus. In another embodiment of a previous aspect, the method further involves selecting compounds that treat or prevent at least one symptom of Parkinson's disease. Suitable animals for use in methods of the invention include mammals (e.g., rodents, rabbits) and invertebrates (e.g., C. elegans and Drosophila). In still other embodiments, the method further comprises selecting a compound that reduces the severity of or delays the onset of a Parkinson's disease symptom in the animal by at least about 10%, 25%, 50%, 75% or 100% compared to a control. In still other embodiments, the method is used to confirm that an HDAC inhibitor can prevent or treat Parkinson's Disease (PD). In various embodiments of any previous aspect, the reporter sequence encodes an amino acid sequence that is detectable by a fluorescent, phosphorescent, luminescent, chemiluminescent, or colorimetric assay. Suitable reporter sequences encode a protein selected from the group consisting of luciferase (e.g., derived from a bacterium or an insect, such as American firefly (Photinus pyralis) or Renilla), green fluorescent protein (GFP), red fluorescent protein (RFP), or a fragment or derivative thereof. In other embodiments, the reporter sequence is a derivative of luciferase having reduced stability compared with naturally-occurring luciferas. For example, a luciferase derivative that contains a mutation that results in the loss of a mammalian transcription factor binding site, optimization of codon usage, or the addition of a degradation sequence (e.g., PEST, ARE (AU-rich element), and CL1).

As will become apparent from the following disclosure, the above-mentioned screen is flexible and can be adapted, as needed, to suit an intended screening paradigm. Thus in one embodiment, the foregoing screen is combined with one or more in vivo assays disclosed herein to further select useful compounds. Preferred in vivo assays use an acceptable animal model of neurological disease (e.g., rodent, rabbit, primate, insect, nematode models). Thus, in one embodiment, the method further includes testing the compound in an acceptable animal model before, during or after exposure to an amount of 1-methyl-4-phenyl-1,2,3,6-tetrahydropyridine (MPTP) or rotenone. Preferably, the amount of MPTP or rotenone is generally sufficient to cause symptoms associated with PD in the animal. Preferred methods pre-screen compounds for suitable activity in one or more of the in vitro screens of the invention. Practice of the invention is compatible with testing compounds either alone as a sole active agent or in combination with other active compounds such as those currently in use to treat certain neurological disorders, such as PD and Huntington's Disease (HD). The use of multiple detection formats (i.e., a combination of in vitro assay, a combination of in vivo assays, or a combination of both in vitro and in vivo assays) with a single candidate compound can extend the selectivity and sensitivity of the detection desired.

Such broad spectrum testing provides advantages such as increasing the chances of detecting compounds with therapeutic activity. This is especially useful when large compound batches are to be analyzed. For instance, and as disclosed below, such candidate compounds can be derived from available compound libraries or can be made using standard synthetic methods including combinatorial-type chemistry manipulations and then tested in accord with the invention.

Notwithstanding the ability to combine various assays of the invention, it will often be useful to focus at least initial efforts on testing large numbers of compounds in vitro. High-throughput assay formats will often be useful as well. In these embodiments, it will often be useful to make recombinant cell lines that include expression vectors that can be used in accord with the invention to detect compound induced changes in at least one of DJ-1, Parkin, and Pink-1 gene activity.

Accordingly, and in another aspect, the invention provides a suitable expression vector that includes at least one of a DJ-1, Parkin or Pink-1 promoter sequence operably linked to at least one reporter sequence of the vector. Preferred vectors will include one or two of such promoter sequences with one of such promoter sequences (e.g., DJ-1, Parkin or Pink-1). Amounts of promoter sequence to employ in each vector will vary, depending on recognized parameters, such as the level of assay sensitivity required and maximizing efficiency of certain recombinant manipulations, such as cell transformation. In general, the vectors will include less than about 4000 base pairs “upstream” of the transcription start site of the DJ-1, Parkin or Pink-1 genes.

In general, further sequence from the DJ-1, Parkin and/or Pink-1 genes will not be needed to make and use the expression vectors of the invention. In some embodiments, it may be useful to include additional sequence from these genes. Such sequence, if needed at all, may improve assay sensitivity and selectivity. In these embodiments, the expression vector will desirably include sequence downstream of the transcription start site of each of the DJ-1, Parkin, or Pink-1 genes, such that the downstream sequence is operably linked to the promoter sequence. Preferably, such downstream sequence information will include less than about 500 base pairs of downstream sequence.

In another aspect, the invention provides recombinant cells and recombinant cell lines that include at least one of the expression vectors disclosed herein. Such cells can derived from primary, secondary, or tertiary sources (cells, tissue) as needed to suit an intended invention objective. Suitable cells lines can be immortalized. As discussed below, the invention is compatible with a wide variety of cells and cell lines, although for many applications cells derived from neuronal, breast, testis, or prostate tissue sources will often be useful. Such cells and cell lines can, in some embodiments, maintain the expression vector transiently. However, in other embodiments, more long term and stable maintenance of the expression vector by the cells will be desirable.

Further provided by the invention is a method for producing the recombinant cell lines provided herein. In one embodiment, the method involves contacting a cell with the expression vector under conditions conducive to introducing the vector into the cell; and transforming the cell to make the recombinant cell line. More specific methods are discussed below.

In another aspect, the invention provides a kit that includes at least one of: at least one of the recombinant cell lines of the invention; and at least one of the expression vectors.

The invention provides compositions and methods for treating a neurological disease. Other features and advantages of the invention will be apparent from the detailed description, and from the claims.

### BRIEF DESCRIPTION OF THE DRAWINGS

FIG. 1 is a schematic drawing showing the −1000DJ-Luc expression vector. The human DJ-1 promoter was linked to a luciferase reporter gene as described in methods.

FIG. 2 is a graph showing activation of the human DJ-1 promoter by H₂O₂.

FIGS. 3A-B are graphs showing activation of the human DJ-1 promoter by certain HDAC inhibitors. Cells stably expressing −1000DJ-Luc were treated with increasing doses of Trichostatin A (TSA (a)) or sodium butyrate (b) for 24 hours.

FIG. 4 is a photograph of a Western blot showing accumulation of endogenous DJ-1 protein in cells treated with HDAC inhibitors. Cells stably expressing −1000DJ-Luc were treated with Trichostatin A (TSA) or sodium butyrate for 24 hours.

FIG. 5 is a graph showing that DJ-1 protects against H₂O₂ and α-synuclein-induced neuronal apoptosis in SH-SY5Y cells.

FIGS. 6A-6D depict the effects of HDAC inhibitors on DJ-1. FIGS. 6A and 6B are graphs showing that HDAC inhibitors, suberoylanilide hydroxamic acid (SAHA), Trichostatin A (TSA), and sodium butyrate (SB) specifically activate luciferase expression directed by the human DJ-1 promoter in SH-SY5Y cells stably expressing the reporter constructs. Values are mean±SEM. n=4. *: P<0.05. FIGS. 6C and 6D are photographs of DJ-1 immunoblots, which show that HDAC inhibitors increase DJ-1 expression in SH-SY5Y cells (FIG. 6C), in primary neuronal cultures containing cortical neurons and glia (FIG. 6D, in first four lanes at left of blot) and in mouse embryonic stem cells (ES) (FIG. 6D, in last three lanes on right of blot) relative to untreated control cells (CTL).

FIG. 7 is a photograph of 2 immunoblots, which show the effects of sodium butyrate (SB) treatment on of DJ-1 and β-actin protein in the cortical and mid-brain tissues from mice injected with control vehicle (CTL) or the HDAC inhibitor sodium butyrate (1200 mg/kg body weight).

FIG. 8 is a graph showing the results of a DJ-1 luciferase assay configured as a high throughput screen. Equal numbers of SH-SY5Y cells stably expressing the 1000DJ-Luc construct were plated in replicate (N=12) with 5 μM pergolide, bromocriptine or SAHA for 24 hours and then assayed for luciferase activity. The assay produced a Z′ value of 0.790 using SAHA as the positive and bromocriptine as the negative control.

### DETAILED DESCRIPTION OF THE INVENTION

The present invention provides compositions and methods for treating a neurological disorder. As described in more detail below, the invention is based in part on the discovery of a screen that can be used to detect compounds that modulate the activity of at least one of the DJ-1, Parkin and Pink-1 genes. Preferred compounds detected by the invention increase the activity of at least one of the genes, typically by increasing promoter function. The invention has a wide spectrum of useful applications, including identifying compounds that can be used to prevent, treat, prolong the onset of or help alleviate symptoms associated with certain neurological disorders.

Illustrative neurological disorders for which the invention can be used to detect new therapeutic compounds (or confirm activity of existing compounds) include those disorders involving the central nervous system (CNS) and particularly the brain. More specific neurological disorders include those known or suspected to impact subcortical structures. More particular neurological disorders in accord with the invention involve at least one of the following medical indications: partial or complete degeneration of nigrostriatal pathway, raphe nuclei, locus ceruleus, and the motor nucleus of vagus; partial or complete degeneration of intrastriatal cortical cholinergic neurons, GABA-ergic neurons. Chemical changes reported to exemplify such indications include, but are not limited to, reductions in at least one of dopamine, serotonin, norepinephrine, choline acetyltransferase, glutamic acid, decarboxylase, and GABA. In some instances, normal aging may be associated with one or more of these abnormal brain characteristics. More specific neurological disorders in accord with the invention include Parkinson's disease (PD) and Huntington's disease (HD). Clinical and behavioral characteristics associated with these and other neurological disorders have been reported. See generally Kandel, E. R. et al. in Principles of Neural Science (Kandel, E. R et al. Eds. 3^{rd} Ed.) Appleton & Lange, Norwalk, Conn.

The invention provides a variety of expression vectors that can be used to make useful recombinant cell lines. Such cell lines can be used in accord with the invention to screen for compounds that can be used therapeutically to help prevent, treat, reduce the severity of, or prolong the onset of a neurological disorder. Thus in one invention aspect, there is provided an expression vector that includes at least one of a DJ-1, Parkin or Pink-1 promoter sequence operably linked to at least one reporter sequence. By “promoter” is meant a polynucleotide sufficient to direct transcription. In one embodiment, a promoter is a DNA sequence that is capable of controlling the transcription of an oligonucleotide sequence into a primary RNA transcript or more particularly mRNA. A promoter is typically located 5′ (i.e., upstream) of an oligonucleotide sequence whose transcription into mRNA it controls, and provides a site for specific binding by RNA polymerase and for initiation of transcription. The term “promoter activity” when made in reference to a nucleic acid sequence (typically a promoter) refers to the ability of the nucleic acid sequence to initiate transcription of an oligonucleotide sequence into mRNA. Preferred promoter sequences are discussed in more detail below.

DNA regions are referred to as “operably linked” when a first polynucleotide is positioned adjacent to a second polynucleotide that directs transcription of the first polynucleotide when appropriate molecules (e.g., transcriptional activator proteins) are bound to the second polynucleotide. For example: a promoter sequence is operably linked to a coding sequence if it controls the transcription of the sequence; a ribosome binding site is operably linked to a coding sequence if it is positioned so as to permit translation. Generally, operably linked means contiguous and, in the case of leader sequences, contiguous and in reading frame.

### Making Expression Vectors

A. Promoter Sequence Information

DJ-1

The sequence of the human and mouse DJ-1 genes is described, for example, by Taira, T. et al. (2001) Gene 263: 285. In particular, the human DJ-1 promoter, gene and 5′-UTR have been disclosed by GenBank as Accession Number AB045294. See the National Center for Biotechnology Information (NCBI)-Genetic Sequence Data Bank (Genbank), National Library of Medicine, 38A, 8N05, Rockville Pike, Bethesda, Md. 20894. See Benson, D. A. et al. (1997) Nucl. Acids. Res. 25: 1 for a description of Genbank.

The sequence disclosed by GenBank as accession no. AB045294 is shown below in Table 1.

TABLE 1

| | |
|---|---|
| 1 | ggatccttct aagctcattc aagaattttg ggctttaact atttcctttg atttaacctg |
| 61 | gtaccaggtg ccaactttag ataataggga tatctaatta cttctaaatt cctcagataa |
| 121 | ggggcctgct tgatggtcac caggtgatct gtgctctcct taagagggaa taagacctag |
| 181 | cgttggcaga gttctgtagg gtgactatag ttaacagtaa tctgttgtat attttaaaat |
| 241 | gttattattg aagagagtaa ctggaatgtt cccagtataa agacaaatgt ttaaggtgat |
| 301 | agagatctca tttaccctga tttaatcatt acacattata tgaaagtatc aaaataccac |
| 361 | atgtacccag aaaacacata cgtctcttac atatcaataa atacaacttg agattatgat |
| 421 | gtaaatacat ctgaccaact tggtacttat tagacttatg tgcgcagcac tgctctagtc |
| 481 | ctgtgggtgc agcagcatca ggatcgttaa agaaaacaaa caatgctgag aaaaaaactc |
| 541 | acacccctga gacatccggg tgtgaataaa tgcggcagag tcgcccgaga tcgggagacc |
| 601 | aggcgtgggg gagaggtccg ggaggcctgg accagagtcc taacagacca gaggcgaaac |
| 661 | gggaaggcgc gccagaaaag gaacaacgca aagggagcag gcgtgcacgg agcgcgaact |
| 721 | aaggaacccc tctgacaacc ccagtccctc ggcagttcca gagaccggct cctcacggag |
| 781 | ggtggcggta gagactgtta agccccgcgg gcgccggggc aggccggact gtgccattcg |
| 841 | tggggggtac catgtgggac cgagccgcct cacccagggc tgtccagcta gaaactcccc |
| 901 | ggtgccaccc ccgcctcagt ccgaggtaga ctcggccgga cgtgacgcag cgtgaggcca |
| 961 | aggcggcgtg agtctgcgca gtgtggggct gagggaggcc ggacggcgcg cgtgcgtgct |
| 1021 | ggcgtgcgtt cactttcagc ctggtgtggg gtgagtggta cccaacgggc cggggcgccg |
| 1081 | cgtccgcagg aagaggcgcg gggtgcaggt cagcgccagc gggggcgcgg cgcatgtgtg |
| 1141 | ggccgtggcg ctgggcggcg tgggggtgct ggacggtgtc cctgtgctgg acggtgtccc |
| 1201 | gctggctcag aaccggcgcg gggcctgggt cggggccgcc ctcgcttccg gcctcccagt |
| 1261 | cgggccctgt cgctggcgtt ggatttgact gaccgccagc gtggtggcaa cgctgaagcg |
| 1321 | tccagaatct tctgcctaac ctctcgccgg catggaactg gctagccgtt ttattaaact |
| 1381 | ctgttttgcg tggacggtaa accctccaga taatctgtaa ataggttaaa aaaaattcgg |
| 1441 | aacctcgttg agctgctgtc gttggcagtg agaactccgc gcagagagac agatgtagtt |
| 1501 | gggttgactt cagtgagggg atttccatct ttctcagtca ttaaaaaaag tgttcagaca |
| 1561 | tttaacactg ttgaccccca cacacaattt tttagtacag ttataactaa gaaaacaaaa |
| 1621 | atcccctcca aaaaattaca agttaattgc gaaagaccac atttaaattt ttgcccatga |
| 1681 | aattcagttt agtcgtttct ctgaaacagt gcttcaaaaa agactgtttc cccgcattgt |
| 1741 | gtgaaatgca ggagacccac gtacttgtat ttttaaaaaa cccatttgca acatactatt |
| 1801 | aaagttggat ttaagagaac atggtagaag aaaatctaag caatactaca ccttttagca |
| 1861 | ccctcattat gttttcatct cagagcaatt aaaactgcta tacaaatcaa cgttaagata |
| 1921 | actaaactgc tgcttttttc gtattcagtt gtctatgaaa accgtttccc taggaagtac |
| 1981 | ttactctgct tgaaaatgct cctaaacttt aaattttggg gtatctcagg gttgcaatga |
| 2041 | aagttttttg aaatcttttt tttttttttt ttttaaggct tgtaaacata taacataaaa |
| 2101 | atggcttcca aaagagctc |

According to the sequence information provided in Table I, the transcription initiation site (start site) of the human DJ-1 gene is at nucleotide position 1016. Accordingly, the sequence provides about 1000 base pairs 5′ to (upstream) in relation to the transcription start site. It further provides about 1100 base pairs 3′ to (downstream) of the transcription start site. By the term “upstream” is meant in the 5′ direction to a particular reference point which in some instances will be a gene transcription start site (i.e., the nucleotide position that begins the primary RNA transcript). Similarly, “downstream” means in the 3′ direction to the particular reference point.

Inactivation of DJ-1 by siRNA sensitizes cells to oxidative stress-induced cell death (Yokota, et al, Biochemical and Biophysical Research Communications, 312: 1342-1348, 2003). In addition, elevated oxidative stress may be a factor in Parkinson's related neurodegeneration. α-Synuclein accumulation increased the generation of toxic hydroxyl free radicals (Hsu et al, Am J Pathol. 157, 401-10, 2000; Xu, et al, Nat Med, 8, 600-06, 2002). In addition, a byproduct of dopamine metabolism in the dopaminergic neurons, H₂O₂, can be converted to a hydroxyl free radical, which makes these cells particularly vulnerable to cell death (Sidhu et al, Faseb J, 18:637-648, 2004). Consistent with these studies, it is believed that overexpression of DJ-1 can block neurotoxicity induced by α-synuclein and H₂O₂ in human dopaminergic cells. Without wishing to be bound by theory, it is also believed that increased expression of neuronal DJ-1 may help to eliminate toxic oxidative stress signals and prevent neuronal cell death.

The DJ-1 promoter harbors a binding site for transcriptional factor SP1, which accounts for the majority of the promoter activity. It is believed that compounds that can induce SP1 or enhance its activity will likely activate DJ-1 expression. The DJ-1 gene has been reported to have other effects. For example, and in addition to exhibiting neuroprotective and anti-oxidative effects, DJ-1 may regulate male fertility and androgen receptor activity, and is a breast cancer marker. The methods of the invention are useful for the identification of agents that can activate or repress DJ-1 gene expression or agents that can regulate various activities modulated by DJ-1. The use of additional cell lines stably expressing luciferase gene directed by the DJ-1 promoter, such as cervical carcinoma HeLa cells or breast cancer MCF cells is useful to differentiate between the tissue specific activities of identified agents. The cell-based luciferase system provided in one embodiment (see the Examples below) is one convenient means provided by this disclosure to identify and to evaluate agents that can transcriptionally regulate a stress-responsive protein important for neurological diseases, nuclear receptor functions, and, possibly, tumorigenesis. Recombinant cells of the invention serve as an efficient and reliable resource to generate leads for drug discovery.

Parkin

The sequence of the human Parkin gene promoter is reported, for example, by West, A. et al. (2001) J. Neurochem. 78: 1146. It is also provided by Genbank as Accession No. AF350258. The Parkin protein and cDNA has been disclosed by Genbank as Accession Nos. BAA25751 and AB009973, respectively.

The sequence disclosed by GenBank as Accession No. AF350258 is shown below in Table 2. It shows, among other things, the Parkin promoter and a partial coding sequence (spanning nucleotide positions 5216 to 5222).

TABLE 2

| | |
|---|---|
| 1 | cttgctggcc ctggggaagt atcttgactt tttttctata agaattggga agctccaaaa |
| 61 | gctctgaata gtgataggag cagaacattg taccagaaag attagtgtaa ttgtactgat |
| 121 | aattgattga gggagtcaac caattgatag gtggatgata ttgtacaagc ctagacaaaa |
| 181 | ggtgatgagg gcacccatta gttcatcgcc acttggtccc ttcatcatta gtacttctct |
| 241 | gccagagaca tctgtttatt tgtattgtaa ttatttaact tgtctctctc cttttcttca |
| 301 | ctaataatgt agcacattta gcactggagc tagacacttc taattatccc ccaatattcc |
| 361 | ttggctacag taataaaaca ttgtgagttt gagccggaca cagagctacc agttaaagac |
| 421 | tacatgtccc agcctctctt gcaactagct gtggccataa gactaggttt tggcaatgga |
| 481 | tttgagcagg agtgaggatt gctgtttctg ggacatgccc tcatagtgaa gctgtttgct |
| 541 | cttcatttct tcctgcctca ttcttgcaga ttgctccata cccatttttc tctcctccac |
| 601 | ctgaagtagg tgttggagat gatgcccttt tggaactaca tagcttcctt catccttttc |
| 661 | ctgagagaca ggtacgtggg cttgggagtt gcttcatggg tcaagctttc ataggctttt |
| 721 | gcaaaaaggg aaaatgtagg tgtatttatt actaggttct ggccagttgg atgagaatga |
| 781 | aagtggggtg ctgtgtctgg gtcatgcaca taatgggaag ctctctgctt ccactttcct |
| 841 | ccttcttgag ggcaggtatt tgaccctggt ggtctcgagc cccccttgtc tcatgtgtca |
| 901 | ttttacagga tgcaaagaac aaacatgctc ctatgcccct gcacctgcct catggggaat |
| 961 | gctgccaacc tcctgggatt gcctacaaaa ttgaacttct attatgagag aaacaacttc |
| 1021 | catcttaatt aagctattgt tatttggtca gcgttacaga tgccaaatta atattttaat |
| 1081 | atataattta taaatattga tgattaccac tacgtgttaa atgagcacat gattggtaaa |
| 1141 | tataaaacac actatacatg taaagtatag gttgaactat cccttatctg aaatgcttgg |
| 1201 | gaccagaagt gttttggatt tgtttttgtt ttttgttcaa atttggaata cagtcatccc |
| 1261 | tcagtctcca tgaggaattg tttccaggac ctcctgcaga taccaaaatc ttcagatgct |
| 1321 | caagtccctg atataacatg gcgtagtatt tgtatgtaac ctacacattt ctacctatat |
| 1381 | actttaaatc atgtctatat tacttataat atctaaaaca atataaatgc tccataaata |
| 1441 | gttgctatgc tctattgttt agggaataat gactcaaaaa aaaaagtctg tacatgttca |
| 1501 | atacagataa aatttttatc ccaaacattt caatccaagg ttagttgaat ccatggaggc |
| 1561 | agaatctgta caaagagctg actatatctg tattatatac tgatggtcac gccaggttct |
| 1621 | gaaaatctga aatccaaaat gctcaaaaga gttttctttg agtgtaatgt caacactcaa |
| 1681 | aaagttttgt attttggacc acttcaggtt tcagactttt ggaataggaa tactcaacct |
| 1741 | ttaatattaa aaaaattatt tattccattc ccctaaactt tgagcataaa gcatctatag |
| 1801 | tcttttgaga aggaaagctg taatagaaag cttaggctga actcaacttc tctgtcacca |
| 1861 | taaccctggg tcgattttct aacttgcttc gtgataagct tgttcagcac agaaagtatc |
| 1921 | tgacaagata aagacaacaa aatactgggt tactagtttc tggtcatggg ctctatcaca |
| 1981 | ccccaaacaa gacttttaaa ggaaaatgaa ctatgaaatc ttcaagttgt gtatctcata |
| 2041 | tctctttata ggcaagcact ataaaaatgt gaatttgaat attatttcat tgcagggctt |
| 2101 | ttgtgatgct tgttttctta tagaatgcaa caaaaatttc atggaaagaa agtctagttt |
| 2161 | ctattgaaga aaaatatttg acattgagat tttaaaaatt ttgccattta catttatcat |
| 2221 | tattttttca ataatcttgc actctcatat ccagattgtt taaataagca tttctgcttg |
| 2281 | cacagcccat ttgatgaaac atatttatta tcaagtttat gtacttgtat cactatcgca |
| 2341 | ctcagagaaa tatcaggagt ctctgtataa ctccttatga tatatacagt tcttcatgtt |
| 2401 | ttaggtggaa gagcattgga atgttgactt atctaactgg aaaagtggtt tggagggtgt |
| 2461 | tggctcttgg agcatgaggt tgcaattaag aaaagctgga aattggcata cacgtcctga |
| 2521 | atcaaaatac accttcagaa agagatgagg acattttcac cttataatgc tgagaagtct |
| 2581 | atactgctaa agataaaaat ggtgaaatgt aaatattggt tatgaaattg aaaattttta |
| 2641 | tttcctgtac cactgaagtt attttgtata aacggtattg tccagccttc tttttatcaa |
| 2701 | gatttgaatg ttgttatttt ggttttcctc ggagtactaa gggcagggac tttgctttgc |
| 2761 | tgatcccaaa tcccagaact gtgcttagca aacactgggt attaaaaaaa aaaaagagag |
| 2821 | agccagttgt tgactgaata aatagatgaa tggataaata atgtttgcat ttaagaatta |
| 2881 | cgatttccaa tggcaagaga ggtattgcta gtacaagatt ttcctttaga acataaaaag |
| 2941 | agaagataat ggatctcaat taagttgttt ataaagaagc ctgcttcata atcaatgttt |
| 3001 | tttttaagtc atgtaggcat acttattaca tttggcaata cagaaacatc agattttgca |
| 3061 | gaactatctc tttaggtgta agattatatt aaagaattaa tatgatacaa gaattatgaa |
| 3121 | tacaggttta ggaaaaaaca gaaaagaacc ccaaccagta aaaaaaaaat taaagtataa |
| 3181 | cattaaaaaa catcaaaatt gtaaatattg tgtagaagaa aaactaaatg attaacctga |
| 3241 | atggttatgg tattgctgat aaatgcatca tcttgactcc taggagaacc aatttatgtg |
| 3301 | aaattccatg aaaaagaatt agttacaaca agcagaattt tagtccattt ccaagaattt |
| 3361 | taactactgt aaatcccctg acacacctcc caaataatta ggatatcgtt ttgcaatagc |
| 3421 | cacatgggaa cctggcccta gaggtctata ggtaatctgt ttcattcatg tattttaagt |
| 3481 | atgtcgttta ggaataagtt atcaggtttg caacctataa gcaaaggaaa taatgtgaca |
| 3641 | ctggaaaaca acactattca tttaacataa tgaattgcca tgtaataact cagatcttcc |
| 3601 | cagggtgtaa attacacaaa tttgaaagat gcatttatta tttaatgcct cattcccaga |
| 3661 | cagttgctta ctcagtagca aaatctgtct tagcatacca agtgtaaagc tatttaacaa |
| 3721 | ataggaaggt ttaaaaaata tatactatca tgcagacagc taaaatattt gtatatattt |
| 3781 | ttaatctttt ttctctaatg atacttagaa tattttattt ttattactac aaataataga |
| 3841 | gatgaaatat gaattgtatt agtagcagag atatatgagc taaagcttgt attgtttaaa |
| 3901 | gcacatcatc ttaaaaggcc tgtcaggaaa cagtgttcat attaagttgg ctttcagtac |
| 3961 | tctaagaaga tgacatcatt ttgtaagaga caagtgttgt tagagcaaat gctaggatat |
| 4021 | tctaaaattt cctaggttga agtgaagaaa tttctcatta tagattattt catgagttta |
| 4081 | tgttcccggt tgtatatcag ctcatgttaa attttgcaag agtttatgat ttctaagaac |
| 4141 | tcaccttcta taaggccctt tgctgagtgg ggctagttag gaattagtaa gtaaagggga |
| 4201 | tcttttttcc tcgtgtaaat agcttaagag taattttggg cggtccagaa accataagtt |
| 4261 | atcaggaagg tgcttataaa tgggcagagt acatcacttg cccaagattc taacaaccta |
| 4321 | gcctgccccc cacacactgt ggggcaccgt ttgctacttg ccaagtaact gccttttttg |
| 4381 | gcaaagacca cccaggacat ggctcagagt ccatcctaag gctggccaac ctctgtaaat |
| 4441 | ctcgtgtccc ctgattcaga gcgagtgcat ttaattcagg aagatcactt acgactgagt |
| 4501 | ttttcatcat ggctttgtct gtgaaaccct cagaaaccag agagtgaggc tggtgcaccg |
| 4561 | ggagcggctg ttgtgccagc agcttggtcc tcttcggcat cttgtctggg catttgttta |
| 4621 | agctcagggt ctctttttct gccaccatct tcctagaaaa tgtcttgttc tcataaaaag |
| 4681 | tgtagtaaaa gaatcagtgg gctttacgga tgtgagcagg aggtctggaa aaaaatatca |
| 4741 | aaaggcgcga taatggtaga aattcaaccc ctcgtagtgc ccaggttgat ccagatgttt |
| 4801 | ggcagctcct aggtgaaggg agctggaccc taggggcggg gcgggaagag ggcaggacct |
| 4861 | tggctagagc tgcaacaagc ttccaaaggt aagcctcccg gttgctaagc gactggtcaa |
| 4921 | cacggcgggc gcatagcccc gccccccggt gacgtaagat tgctgggcct gaagccggaa |
| 4981 | agggcggcgg tggggggctg ggggcaggag gcgtgaggag aaactacgcg ttagaactac |
| 5041 | gactcccagc aggccctggg ccgcgccctc cgcgcgtgcg cattcctagg gccgggcgcg |
| 5101 | ggggcgggga ggcctggagg atttaaccca ggagagccgc tggtgggagg cgcggctggc |
| 5161 | gccgctgcgc gcatgggcct gttcctggcc cgcagccgcc acctacccag tgaccatgat |
| 5221 | aggtacgtgg gta |

PINK-1

The structure and function of the PINK-1 gene is described, for example, by Valente E M, et al. Science (2004) 304(5674):1158-60; and Unoki, M. and Nakamura, Y. in Oncogene (2001) 20: 4457-4465. Gene and protein structure are provided at GenBank Accession Nos. NP_{—}115785 (protein) and NM_{—}032409 (cDNA). Additional information about PINK-1 gene structure can be obtained under Ensembl translation ID No. ENSP00000289840 (peptide product of gene no. ENSG00000158828. For information about the Ensembl database, see Ewan Birney et al. Genome Res. 2004 14: 925-928; Arne Stabenau et al. Genome Res. 2004 14: 929-933; Simon C. Potter et al. Genome Res. 2004 14: 934-941; and Val Curwen et al. Genome Res. 2004 14: 942-950.

It will be appreciated that it is possible to clone additional sequence upstream of the promoter sequences provided in Tables 1 and 2, as well as in the Examples below using conventional methods. Such methods generally include accessing the complete human genome sequence on Genbank (or other databases such as Ensembl), identifying the site on the sequence corresponding to the gene of interest and simply obtaining the additional sequence from the Genbank or Ensembl database. Particular methods for providing such promoter sequence for the Parkin and Pink-1 genes are provided below. It will be apparent that the precise sequence of the DJ-1, Parkin and Pink-1 promoters is not needed to practice the invention so long as at least a “functional portion” of that promoter is included in the expression vectors of the invention.

Preferred functional portions of a given promoter sequence include enough of that sequence to drive transcription of the detectable reporter sequence encoded by the expression vector. In one embodiment, each of the DJ-1, Parkin or Pink-1 promoter sequences include about 2500 base pairs or less upstream of the respective DJ-1, Parkin or Pink-1 transcription start site, for instance, about 1500 or 1000 base pairs upstream of the DJ-1, Parkin or Pink-1 transcription start site or less. Additionally, suitable functional portions of such promoter sequences include those constructs having about 500 base pairs upstream of the respective DJ-1, Parkin or Pink-1 transcription start site e.g., about 100 or about 50 or less base pairs upstream of the DJ-1, Parkin or Pink-1 transcription start site. In many instances, the functional portion of the DJ-1, Parkin and Pink-1 promoter sequences will span between about 200 base pairs to about 2500 base pairs upstream of the respective transcription start site, such as 250 to about 1500 base pairs.

A suitably functional portion of the DJ-1, Parkin or Pink-1 promoters is, in one embodiment, a component of the presently claimed expression vectors. The term “vector” means any nucleic acid sequence of interest capable of being incorporated into a host cell and resulting in the expression of a nucleic acid sequence of interest. Vectors can include, e.g., linear nucleic acid sequences, plasmids, cosmids, phagemids, and extrachromosomal DNA. In many embodiments, the vector will be a plasmid or related sequence that is replicable in bacteria. Specifically, the vector can be a recombinant DNA. Also used herein, the term “expression” or “gene expression” is meant to refer to the production of the detectable reporter sequence including transcription of its DNA and translation of its RNA transcript. Suitable expression vectors in accord with the invention will include, for instance, a “cloning site” that will be understood to include at least one restriction endonuclease site. Typically, multiple different restriction endonuclease sites (e.g., a polylinker) are contained within the nucleic acid.

The term “expression vector” including plural forms, means vectors capable of expressing a nucleic acid molecule or polypeptide sequence in a cell. Exemplary vectors include at least one of a DJ-1, Parkin, or Pink-1 gene promoter (or functional portion thereof) linked to an expressible reporter sequence. Transcriptional activation of the promoter sequence is registered by expression of the reporter sequence, which typically encodes a detectable amino acid sequence as provided herein.

Conventional procedures were also used to make vector DNA, cleave DNA with restriction enzymes, ligate and purify DNA, transform or transfect host cells, culture the host cells, and isolate and purify proteins and polypeptides. See generally Sambrook et al., Molecular Cloning (2d ed. 1989), and Ausubel et al. in Current Protocols in Molecular Biology, John Wiley & Sons, New York (1989). Additional promoter sequences encompassed by the invention include those sequences that can hybridize under “high stringency” conditions to one of the sequences provided in Tables I and II. Such high stringency conditions are known in the field and include, but are not limited to, hybridization conditions involving a wash at about 65° C. in 0.1×SSC (or 0.1.×.SSPE). See Sambrook et al., supra, for more information relating to performing a high stringency hybridization. Additional promoter sequences encompassed by this disclosure includes a promoter sequence or functional portion that is at least 80% identical to one of the nucleic acid sequences shown in Tables I and II. Preferably, such sequences will be at least about 90% identical, more preferably at least about 95% identical with at least about 99% identical being useful for many screening applications.

Unless otherwise specified, percent sequence identity of two nucleic acids is determined using the algorithm of Karlin and Altschul (1990) PNAS USA 87:2264-2268, modified as in Karlin and Altschul (1993) PNAS USA 90:5873-5877. Such an algorithm is incorporated into the NBLAST and XBLAST programs of Altschul et al (1990) J. Mol. Biol. 215:402-410. BLAST nucleotide searches are performed with the NBLAST program, score=100, word length=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (NBLAST and XBLAST) are used. More particular examples of such suitable promoter sequences include those having gaps (i.e., contiguous or non-contiguous deletions) in the sequences shown in Tables 1 and II as well as certain nucleotide substitutions, additions, and deletions. More particular expression vectors in accord with the invention will generally also include sequence 3′ (downstream) from the transcription start site of at least one of the DJ-1, Parkin, and Pink-1 genes. Typically, the size or length of such a sequence will be less than about 1000 kb, typically between about 20 base pairs to about 500 base pairs.

Thus in one embodiment, an expression vector of the invention further includes a DJ-1, Parkin or Pink-1 downstream sequence having about 250 base pairs downstream from the respective human DJ-1, Parkin or Pink-1 transcription start site, e.g., about 100 base pairs, about 50 base pairs, or about 25 base pairs. Preferred downstream sequences are typically operably linked to the promoter sequence or functional portion thereof. Such linkage can be direct (i.e., direct covalent attachment) or be indirect, such as when the downstream sequence is spaced from the promoter sequence by a spacer element (having e.g., less than about 50 base pairs (eg., less than 10 base pairs). However in most embodiments, the promoter sequence will not be spaced at all from the downstream sequence. Further downstream sequences acceptable for use with the invention include those that hybridize to any one of the sequences shown in Table 1 and 2 under high stringency conditions. More preferred downstream sequences are those that are at least 80% identical to one of the nucleic acid sequences shown in Tables I and II. Preferably, such sequences will be at least about 90% identical, more preferably at least about 95% identical with at least about 99% identical being useful for many screening applications.

Accordingly, in one embodiment, the invention provides a particular expression vector that includes a human DJ-1 promoter sequence including between from about 50 base pairs to 1000 base pairs upstream of the human DJ-1 transcription start site. The promoter sequence further comprising linked in sequence a DJ-1 downstream sequence, having about 200 base pairs or less downstream of the human DJ-1 transcription start site. Preferably, the DJ-1 downstream sequence is about 65 base pairs downstream of the human DJ-1 transcription start site.

In another particular embodiment, the invention provides an expression vector that includes a human Parkin promoter sequence comprising between from about 50 base pairs to 1000 base pairs upstream of the human Parkin transcription start site. Preferably, the promoter sequence further includes an operably linked Parkin downstream sequence having about 50 base pairs to 200 base pairs downstream of the human Parkin transcription start site. Preferably, the Parkin downstream sequence is about 100 base pairs or less downstream of the human Parkin transcription start site.

According to still another particular embodiment, the expression vector includes a human Pink-1 promoter sequence comprising between from about 50 base pairs to 1000 base pairs upstream of the human Pink-1 transcription start site, the promoter sequence further comprising linked in sequence a Pink-1 downstream sequence having about 50 base pairs to 200 base pairs downstream of the human Pink-1 transcription start site. Preferably, the Pink-1 downstream sequence is about 100 base pairs or less downstream of the Pink-1 gene transcription start site.

In addition to the foregoing promoter sequences, the expression vectors will have one or a combination of other components needed to achieve the objects of the invention.

B. Reporter Sequences and Expression Vectors

In particular, most expression vectors will feature a detectable reporter sequence (e.g., luciferase, chloramphenicol transferase, beta-galactosidase) that preferably enables determination of the presence of (and if needed the amount of) transcription from the vector. It is an object of the invention to detect compounds that modulate (preferably increase) transcription from such vectors. The product of the reporter gene may be nearly any detectable molecule, such as the following biosensors: luciferin (luciferase substrate); aequorin; Fluo-3/acetoxymethyl (esterase substrate); FDG (β-gal substrate); or CCF2, which is a β-lactamase substrate. See generally, J. E. Gonzalez and P. A. Negulescu, Curr. Opin. Biotechnol. 9, 624 (1998). In some embodiments, it will be useful to have a detectable reporter sequence (gene) that encodes a fluorescent, phosphorescent, luminescent, or chemiluminescent protein whose expression can be distinguished from that of other cell components via conventional methods. In some embodiments however, it may be useful to employ reporter sequences that encode proteins detectable by calorimetric methods. Preferably, the amino acid sequence encoded by the detectable reporter sequence is directly detectable by the assay of the invention. Examples of such suitable sequences include those encoding luciferase, green fluorescent protein (GFP), red fluorescent protein (RFP); as well as fluorescent fragments and derivatives thereof. See e.g., U.S. Pat. Nos. 6,146,826; 5,741,668; 5,804,387; 6,723,537 and 6,391,630. In embodiments in which luciferase is the detectable amino acid sequence of choice, the enzyme is preferably derived from a bacterium or an insect such as the American firefly (Photinus pyralis) or Renilla. Examples of suitable luciferase, GFP and RFP fragments have been disclosed in the U.S. Pat. Nos. 6,146,826; 5,741,668; 5,804,387; 6,723,537; 6,391,630; and references cited therein.

More particular expression vectors for use with the invention will include a detectable reporter sequence the encodes what is referred to herein as a “derivative” of the luciferase enzyme i.e., a luciferase that has reduced intracellular stability compared with a naturally-occurring luciferase. Such luciferase derivatives are well-known in the field and include commercially available enzyme derivatives. Preferred luciferase derivatives generally include a genetic mutation that provides to the enzyme one or more of the following characteristics: loss of a mammalian transcription factor binding site, optimization of codon usage, addition of a degradation sequence (e.g., at least one of PEST, ARE (AU-rich element), and CL1 element). By the term “PEST” is meant the forty-amino acid sequence isolated from the C-terminal of mouse ornithine carboxylase. See Li, X. (1998) J. Biol. Chem. 273: 34970. By “CL1” is meant a reported degradation signal. See Gilon, T. et al. (1998) EMBO J. 17: 2759. By “ARE” is meant a disclosed AU-rich element. Fan, X. C. et al. (1997) Genes Dev. 11:2557.

More preferred luciferase derivatives and vectors encoding the same are commercially available from Promega Corporation (Madison, Wis.). See Promega Technical Manaual 242 entitled Rapid Response™ Vectors (December, 2003 version) pp. 1-20 (hereinafter “Promega Technical Manual”). The following Promega vectors are preferred for many invention embodiments: pGL3(R2.1); pGL3(R2.2); phRG(R2.1); and phRG(R2.2). Each of these specific vectors can be conventionally manipulated to include at least one of the DJ-1, Pink-1, and Parkin promoter sequences (including functional fragments) that are disclosed herein.

As should be apparent, the invention is compatible with a broad spectrum of specific expression vector constructs. In addition to the aforementioned DJ-1, Pink-1, and Parkin promoter sequences (including functional fragments) and detectable reporter sequences, expression vectors of the invention may include additional elements that, for instance, support replication in a microbial host. In this embodiment, the expression vector will include a suitable origin of replication recognized by the intended microbial host and optionally, a promoter which promoter, which will function in the host and a phenotypic selection gene such as a gene encoding proteins conferring antibiotic resistance or supplying an autotrophic requirement. Similar constructs will be manufactured for other hosts. E. coli is typically transformed using pBR322. See Bolivar et al., Gene 2, 95 (1977). pBR322 contains genes for ampicillin and tetracycline resistance and thus provides easy means for identifying transformed cells.

More particular expression vectors in accord with the invention include at least one of the following components: 1) SV40 late poly(A) site; 2) ColE1-derived origin of replication; and 3) β-lactamase or functional fragment thereof. In embodiments in which more than one of the foregoing components is present, each will be operably linked to the nucleic acid encoding the detectable reporter sequence. Further specific vectors will further include an f1 origin of replication and an upstream synthetic poly(A) region operably linked to the reporter sequence. Preferred sequences for these elements have been reported. See the Promega Technical Manual, for instance.

Nearly any suitable phenotype selection gene (also known as a drug resistance gene) or a functional fragment thereof can be encoded by the expression vector. Examples of suitable genes include, but are not limited to, neomycin, hypoxanthine phosphoribosyl transferase, puromycin, dihydrooratase, glutamine synthetase, histidine D, carbamyl phosphate synthase, dihydrofolate reductase, multidrug resistance 1 gene, aspartate transcarbamylase, xanthine-guanine phosphoribosyl transferase, adenosine deaminase; or a functional fragment thereof. In a particular invention embodiment suitable for many invention applications, the expression vector will include at least one of and preferably all of the following components operably linked in sequence:
1) a polynucleotide encoding an ampicillin resistance gene or functional fragment thereof,
2) an f1 origin sequence,
3) an upstream synthetic poly(A) region,
4) the DJ-1, Parkin, or Pink-1 promoter sequence (or functional fragment thereof) covalently linked to a corresponding downstream sequence as provided herein,
5) a polynucleotide sequence encoding a luciferase derivative,
6) an SV40 late poly (A) signal; and
7) a polynucleotide encoding a neomycin resistance gene; or functional fragment thereof.

In a more specific embodiment, expression vector components (1)-(3) and (5)-(7) are provided by vectors disclosed by the Promega Technical Manual. Component (4) is preferably provided by anyone of the DJ-1, Parkin or Pink-1 promoter sequences provided herein which sequence is preferably linked to a corresponding downstream sequence, also as provided herein. Although the nucleotide length of component (4) of the expression vector will vary depending, for instance, on intended use, in most cases the length will be less than about 5000 base pairs, preferably less then about 4000 or 3000 base pairs, more preferably between from about 500 to about 2500 base pairs.

Thus in particular invention embodiments described below in the Examples, there is provided: 1) a first expression vector that includes expression vector components (1)-(3) and (5)-(7), a DJ-1 promoter sequence having of about 1000 base pairs and about 65 base pairs of downstream sequence; 2) a second expression vector that includes expression vector components (1)-(3) and (5)-(7), a Parkin promoter sequence of about 1488 base pairs and about 68 base pairs of downstream sequence; and 3) a third expression vector that includes expression vector components (1)-(3) and (5)-(7); and a Pink-1 promoter sequence having about 1994 base pairs and a downstream sequence of about 32 base pairs.

Making Cells for Use in Screens

As discussed, the invention is flexible and can be used to screen with a wide variety of cells and cell lines that have been transformed by one or a combination of the expression vectors provided herein. Suitable cells and cell lines are generally eukaryotic and can be transformed by the expression vector. A number of types of cells may act as suitable host cells for the expression vector. Mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

However, in most cases it will be useful to transfect or transform cells that are known or suspected of having cell factors that can, under appropriate conditions, modulate at least one of the DJ-1, Parkin or Pink-1 promoter sequences provided herein. It is thus an object of the invention to screen candidate compounds that have potential to modulate (increase or decrease) the activity of such cell factors which modulation is detectable by the reporter sequence. More particular examples of cells that are believed to harbor cell factors that can interact with one or more of the DJ-1, Parkin or Pink-1 promoter sequences include those obtained directly from (e.g., a primary cell culture) or derived from the following specific tissues: breast, prostate, testis, neurons, glia, colon, pancreas, stomach, esophagus, astrocytes, lung, lymph, skin; as well as immortalized cell lines obtained from such tissues. A wide variety of such cells and cell lines are readily obtained from the American Type Culture Collection (ATCC, Manassas, Va. (USA)).

The following Tables III-VII provide illustrative cells for use with the invention that can be obtained from the ATCC. In particular, Table III provides illustrative non-tumor, neuronal-like cells; tumor-derived neuronal-like cells, glioblastoma cells, medulloblastome-derived cells; retinoblastoma-derived cells; and neuroendocrine tissue; Table IV provides exemplary tumor cell lines; Table V provides various mammary gland derived cells lines; Table VI provides illustrative prostate derived cells lines; and Table VII provide examples of testis-derived cell lines.

TABLE III

| ATCC No. | Species | Name | Tissue Source |
|---|---|---|---|
| CRL-10442 | human | HCN-1A | brain |
| CRL-10742 | human | HCN-2 | brain |
| CCL-127 | human | IMR-32 | brain; neuroblastoma |
| CRL-1718 | human | CCF-STTG1 | brain; astrocytoma |
| CRL-2060 | human | PFSK-1 | brain; cerebellum; |
| | | | malignant primitive |
| | | | neuroectodermal tumor |
| CRL-2137 | human | SK-N-AS | brain; neuroblastoma |
| CRL-2142 | human | SK-N-FI | brain; neuroblastoma |
| CRL-2149 | human | SK-N-DZ | brain; neuroblastoma |
| CRL-2266 | human | SH-SY5Y | brain; neuroblastoma |
| CRL-2267 | human | BE(2)-M17 | brain; neuroblastoma |
| CRL-2268 | human | BE(2)-C | brain; neuroblastoma |
| CRL-2270 | human | MC-IXC | brain; neuroblastoma |
| CRL-2271 | human | SK-N-BE(2) | brain; neuroblastoma |
| CRL-2273 | human | CHP-212 | brain; neuroblastoma |
| CRL-8621 | human | SVGp12 | brain |
| HTB-10 | human | SK-N-MC | brain; |
| | | | neuroepithelioma, |
| | | | metastic site: |
| | | | supra-orbital area |
| HTB-11 | human | SK-N-SH | brain; neuroblastoma, |
| | | | metastic site: bone |
| | | | marrow |
| HTB-12 | human | SW 1088 | brain; astrocytoma |
| HTB-13 | human | SW 1783 | brain; astrocytoma |
| HTB-15 | human | U-118 MG | brain; glioblastoma; |
| | | | astrocytoma |
| CRL-1620 | human | A172 | brain; glioblastoma |
| CRL-1690 | human | T98G | brain; glioblastoma |
| | | | multiforme |
| CRL-2020 | human | DBTRG-05MG | brain; glioblastoma |
| CRL-2365 | human | M059K | brain; malignant |
| | | | glioblastoma; glioma |
| CRL-2366 | human | M059J | brain; malignant |
| | | | glioblastoma; glioma |
| CRL-7773 | human | TE 615.T | brain; |
| | | | ganglioneuroblastoma |
| HTB-138 | human | Hs 683 | brain; glioma |
| HTB-14 | human | U-87 MG | brain; glioblastoma; |
| | | | astrocytoma |
| HTB-148 | human | H4 | brain; neuroglioma |
| HTB-16 | human | U-138 MG | brain; glioblastoma |
| CRL-8805 | human | TE671 | brain; cerebellum; |
| | | subline No. 2 | medulloblastoma |
| HTB-185 | human | D283 Med | brain; cerebellum; |
| | | | medulloblastoma, |
| | | | matastic site: |
| | | | peritoneum |
| HTB-186 | human | Daoy | brain; cerebellum; |
| | | | desmoplastic cerebellar |
| | | | medulloblastoma |
| HTB-187 | human | D341 Med | brain; cerebellum; |
| | | | medulloblastoma |
| HTB-169 | human | WERI-Rb-1 | retinoblastoma; eye; |
| | | | retina |
| HTB-18 | human | Y79 | retinoblastoma; eye; |
| | | | retina |
| CRL-5813 | human | NCI-H660 | lung; carcinoma; |
| | | | small cell lung |
| | | | cancer extrapulmonary |
| | | | origin (prostate), |
| | | | metastic site: |
| | | | lymph node |
| CRL-5893 | human | NCI-H1770 | lung; carcinoma; non- |
| | | | small cell lung cancer; |
| | | | metastic site: lymph |
| | | | node |
| CRL-2139 | human | SK-PN-DW | malignant primitive |
| | | | neuroectodermal |
| | | | tumor; retroperitoneal |
| | | | embryonal tumor |
| CRL-1973 | human | NTERA-2 c1.D1 | malignant pluripotent |
| | | | embryonal carcinoma; |
| | | | testis, metastic site: |
| | | | lung |

TABLE IV

| ATCC No. | Name | Cancer Type | Tissue Source |
|---|---|---|---|
| CRL-7365 | Hs 605.T | carcinoma | mammary gland; breast |
| CRL-7368 | Hs 606 | carcinoma | mammary gland; breast |
| HTB-126 | Hs 578T | ductal carcinoma | mammary gland; breast |
| CRL-2320 | HCC1008 | ductal carcinoma | mammary gland; breast |
| CRL-2338 | HCC1954 | ductal carcinoma | mammary gland; breast |
| CRL-7345 | Hs 574.T | ductal carcinoma | mammary gland; breast |
| CRL-2314 | HCC38 | primary ductal | mammary gland; breast |
| | | carcinoma | |
| CRL-2321 | HCC1143 | primary ductal | mammary gland; breast |
| | | carcinoma | |
| CRL-2322 | HCC1187 | primary ductal | mammary gland; breast |
| | | carcinoma | |
| CRL-2324 | HCC1395 | primary ductal | mammary gland; breast |
| | | carcinoma | |
| CRL-2331 | HCC1599 | primary ductal | mammary gland; breast |
| | | carcinoma | |
| CRL-2336 | HCC1937 | primary ductal | mammary gland; breast |
| | | carcinoma | |
| CRL-2340 | HCC2157 | primary ductal | mammary gland; breast |
| | | carcinoma | |
| CRL-2343 | HCC2218 | primary ductal | mammary gland; breast |
| | | carcinoma | |
| CRL-7482 | Hs 742.T | scirrhous | mammary gland; breast |
| | | adenocarcinoma | |

TABLE V

| Species | Cell Line Name | ATCC No. | Description |
|---|---|---|---|
| human | MCF 10A | CRL-10317 | fibrocystic disease |
| human | MCF 10F | CRL-10318 | fibrocystic disease |
| human | MCF-10-2A | CRL-10781 | fibrocystic disease |
| human | MCF-12A | CRL-10782 | |
| human | MCF-12F | CRL-10783 | |
| human | Hs 564(E).Mg | CRL-7329 | |
| human | Hs 565(A).Mg | CRL-7330 | cyst |
| human | Hs 565(D).Mg | CRL-7333 | cyst |
| human | Hs 579.Mg | CRL-7347 | |
| human | Hs 617.Mg | CRL-7379 | |
| human | Hs 873.T | CRL-7610 | abnormal |
| human | Hs 874.T | CRL-7611 | abnormal |
| human | Hs 875.T | CRL-7612 | abnormal |
| human | Hs 877.T | CRL-7613 | abnormal |
| human | Hs 879(B).T | CRL-7615 | |
| human | Hs 880.T | CRL-7616 | abnormal |
| human | Hs 885.T | CRL-7618 | abnormal |
| human | Hs 912.T | CRL-7661 | abnormal |
| human | Hs 938.T | CRL-7688 | abnormal |
| human | SW527 | CRL-7940 | Paget's disease |
| human | 184A1 | CRL-8798 | epithelium; |
| | | | chemically |
| | | | transformed |
| human | 184B5 | CRL-8799 | epithelium; |
| | | | chemically |
| | | | transformed |

TABLE VI

| Species | Cell Line Name | ATCC No. | Description |
|---|---|---|---|
| human | RWPE-1 | CRL-11609 | transfected with Ki-MSV |
| human | RWPE-2 | CRL-11610 | transfected with HPV-18 |
| | | | and Ki-MSV |
| human | PWR-1E | CRL-11611 | immortalized with Ad12- |
| | | | SV40 hybrid virus |
| human | PZ-HPV-7 | CRL-2221 | epithelium; HPV-18 |
| | | | transformed |

TABLE VII

| Species | Cell Line Name | ATCC No. |
|---|---|---|
| human | Hs 1.Tes | CRL-7002 |
| human | Hs 181.Tes | CRL-7131 |

In one example of an appropriate neuroblastoma cell for use with the invention is human neuroblastoma cell SH-SY5Y as mentioned in the Examples.

The cells and cell lines disclosed herein can be transfected with one or more of the expression vectors already described. Typically, just one type of expression vector will be used to transfect the cells. The term “transfection” as used herein means an introduction of a foreign DNA or RNA into a cell by mechanical inoculation, electroporation, infection, particle bombardment, microinjection, or by other known methods. Alternatively, one or a combination of expression vectors can be used to transform the cells and cell lines. The term “transformation” as used herein means a stable incorporation of a foreign DNA or RNA into the cell, which results in a permanent, heritable alteration in the cell. A variety of suitable methods are known in the field and have been described. See e.g., Ausubel et al, supra; Sambrook, supra; and the Promega Technical Manual.

In particular invention embodiments, a cell or cell line of choice is manipulated so as to be stably transformed by an expression vector of the invention. In some invention embodiments, transient expression of the vector (e.g., for less than about a week, such as one or two days) will be more helpful. Cells and cell lines that are transiently transfected or stably transformed by one or more expression vectors disclosed herein will sometimes be referred to as “recombinant”. By the phrase “recombinant” is meant that the techniques used for making cell or cell line include those generally associated with making and using recombinant nucleic acids (e.g., electroporation, lipofection, use of restriction enzymes, ligases, etc.).

The invention also provides methods for detecting and in some cases analyzing compounds that increase activity of one or more of the DJ-1, Parkin, and Pink-1 promoters (or functional portions thereof). Certain of those compounds can be further selected if needed to identify those with therapeutic capacity to treat or prevent the above-described neurological conditions. Preferred detection and analysis methods include both in vitro and in vivo assays to determine the therapeutic capacity of agents to prevent, treat, prolong the onset of, or help alleviate the symptoms of such disorders.

### Screening Assays

As discussed, typical screening assays according to the invention include at least one of and preferably all of the following steps: (a) contacting a recombinant cell or cell line made in accord with the invention with at least one candidate compound; (b) incubating the cells under conditions sufficient to express a detectable reporter sequence; and (c) detecting a change in the expression of the reporter sequence (relative to a suitable control) as being indicative of the presence (or absence) of a compound that can modulate one or more of the DJ-1, Parkin and Pink-1 gene promoters. Preferred compounds will be useful to prevent, treat, prolong the onset of, or help reduce symptoms associated with a neurological disorder, such as PD and/or HD.

A more particular embodiment of the forgoing screening assay features all of the following steps:
1) preparing a population of recombinant cells that include at least one of (preferably less then three, usually one of) the expression vectors (e.g., a DJ-1, Parkin and Pink-1 gene promoter operably linked to a reporter gene) disclosed herein;
2) adding about 0.01 to about 2000 micromoles of a known or candidate compound to the cells under conditions sufficient to express a detectable reporter sequence (e.g., luciferase, GFP or RFP);
3) measuring activity of the detectable reporter sequence; and
4) determining the effect of the known or candidate compound on the measured activity in which an increase (relative to a suitable control) is taken to be indicative that the a compound that can enhance activity of at least one of the DJ-1, Parkin, and Pink-1 promoter sequence.

This general assay can effectively measure the capacity of the candidate compound to modulate the DJ-1, Parkin or Pink-1 promoter sequence. The recombinant cells can stably express the vector or such expression may be transient. References herein to a “standard in vitro screening assay,” or similar phrases, refers to the above protocol of steps 1) through 4). Suitable cells for conducting the assay have already been disclosed. Although it is generally preferred that whole cells be used in the assay, some embodiments may be practiced with a lysate of such cells or tissue, or a substantially purified fraction of the lysate may be employed in some cases.

As discussed herein, mutations in the DJ-1, Parkin and Pink-1 genes are associated with Parkinson's disease. Altering the expression of these genes is likely to be useful not only for the treatment of Parkinson's Disease, but also in the treatment of other neurological disorders, particularly disorders associated with a decrease in the expression of a DJ-1, Parkin or Pink-1 gene, or with excess neuronal cell death. Compositions of the invention are useful for the high-throughput low-cost screening of candidate compounds to identify those that modulate the expression of a DJ-1, Parkin or Pink-1 polypeptide or nucleic acid molecule. In one embodiment, the effects of known therapeutic drugs on the expression of a DJ-1, Parkin or Pink-1 gene can be assayed using microarrays of the invention. Tissues or cells treated with these drugs are compared to untreated corresponding control samples to produce expression profiles of known therapeutic agents. Knowing the identity of sequences that are differentially regulated in the presence and absence of a therapeutic agent is useful in understanding the mechanisms of drug action.

Any number of methods are available for carrying out screening assays to identify new candidate compounds that promote the expression of a DJ-1, Parkin or Pink-1 gene. In one working example, candidate compounds are added at varying concentrations to the culture medium of cultured cells expressing one of the nucleic acid sequences of the invention. Gene expression is then measured, for example, by microarray analysis, Northern blot analysis (Ausubel et al., supra), reverse transcriptase PCR, or quantitative real-time PCR, using any appropriate fragment prepared from the nucleic acid molecule as a hybridization probe. The level of gene expression in the presence of the candidate compound is compared to the level measured in a control culture medium lacking the candidate molecule. A compound that promotes an increase in the expression of a DJ-1, Parkin or Pink-1 gene a DJ-1, Parkin or Pink-1 gene, or a functional equivalent thereof, is considered useful in the invention; such a molecule may be used, for example, as a therapeutic to treat a neurological disorder in a human patient.

In another working example, the effect of candidate compounds may be measured at the level of polypeptide production using the same general approach and standard immunological techniques, such as Western blotting or immunoprecipitation with an antibody specific for a polypeptide encoded by a DJ-1, Parkin or Pink-1 gene. For example, immunoassays may be used to detect or monitor the expression of at least one of the polypeptides of the invention in an organism or in a cell in culture. Polyclonal or monoclonal antibodies that are capable of binding to such a polypeptide may be used in any standard immunoassay format (e.g., ELISA, Western blot, or RIA assay) to measure the level of the polypeptide. In some embodiments, a compound that promotes an increase in the expression or biological activity of the polypeptide is considered particularly useful. Again, such a molecule may be used, for example, as a therapeutic to delay, ameliorate, or treat a neurological disorder (e.g., a disorder characterized by excess cell death) in a human patient.

### Test Compounds and Extracts

The standard in vitro screening assay is flexible and can be used to screen one or a combination of different compounds. Illustrative examples follow and include, but are not limited to chemical libraries. In embodiments in which large scale screening is desirable, the methods of the invention can be used to screen, for instance, publicly available chemical libraries. Such libraries include the following: Chem Bridge DiverSet E (16,320 compounds, ChemBridge Corp. San Diego, Calif.); Bionet 1 (4,800 compounds; Ryan Scientific, Isle of Palms, S.C.); CEREP library (4,800 compounds; CEREP, Richmond, Wash.). Further chemical libraries may be obtained from the U.S. National Cancer Institute such as the Structural Diversity Set, version 2 (2,000 compounds); Mechanistic Diversity Set (900 compounds); Open Collection 1 (90,000 compounds), and Open Collection 2 (10,000 compounds). Another compound library that can be used in accord with the invention can be obtained from the U.S. National Institute of Neurological Disorders and Stroke (NINDS) and is called the NINDS Custom Collection (1,040 compounds). Further chemical library collections can be used with the invention including those extracts obtained from various plants, fungi and marine sources available from the U.S. National Cancer Institute. Another compound library that can be used with the invention is the Prestwick Chemical Library (available from Prestwick Chemical, Inc.; Washington D.C). The Prestwick library has been reported to include compounds with known efficacy in different therapeutic areas. In particular, certain compounds in the library have accepted activity in neuropsychiatry, as anti-diabetics, antivirals, antihypertensives, antipyretics, anti-inflammatory drugs, as well as antibiotics and other anti-infectives.

Further compounds may be screened according to the invention have been reported and include histone deacetylase (HDAC) inhibitors. Examples of such compounds have been disclosed in the following references: U.S. Patent Publication Nos. 2004/0087657; 2004/0077591; 2004/0087652; 2004/0087657; U.S. Pat. Nos. 6,541,661; 6,720,445; 5,369,108; 5,700,811; 5,773,474; 5,055,608; 5,175,191; Bioassays 17, 423-430 (1995), Saito, A., et al., PNAS USA 96, 4592-4597, (1999), Furamai R. et al., PNAS USA 98 (1), 87-92 (2001), Komatsu, Y., et al., Cancer Res. 61(11), 4459-4466 (2001), Su, G. H., et al., Cancer Res. 60, 3137-3142 (2000), Lee, B. I. et al., Cancer Res. 61(3), 931-934, Suzuki, T., et al., J. Med. Chem. 42(15), 3001-3003 (1999); published PCT Application WO 01/18171; and published Japanese Patent Application No. 2001-348340; the disclosures of which are incorporated herein by reference. Additionally preferred HDAC compounds are short-chain fatty acid, hydroxamic acid, cyclic tetrapeptide, or benzamides, for instance, 4-phenylbutyrate, valproic acid, suberoylanilide hydroxamic acid (SAHA), pyroxamide, trochostatin A, oxamflatin, trapoxin A, apicidin, butyrate salt; or a derivatives thereof.

Regarding HDAC inhibitors, there are some reports that the transcriptional activity of SP1 can be increased by acetylation of the lysine residues. Several members of histone deacetylase (HDAC) inhibitors, which are thought to help promote the acetylation of transcription factors as well as chromosome-binding histones, have been reported to protect against neuronal cell death through an SP1-dependent pathway. Furthermore, these same HDAC inhibitors have been disclosed as alleviating motor symptoms in animal models of another progressive neurodegenerative disorder, Huntington's disease. In addition to demonstrate neuroprotective effects, HDAC inhibitors are understood by some to induce apoptosis in tumor cells. Currently, several HDAC inhibitors are being tested in phase I and II clinical trials to treat various cancers due to their efficacy and low toxicity. Thus, HDAC inhibitors represent one attractive compound family that can be used as a source of particular compounds in the screening assays provided herein.

The known or candidate compounds, including HDAC inhibitors, can be employed as a sole active agent or in combination with other agents, including other compounds to be tested. In most instances, the in vitro assays are performed with a suitable control assay usually comprising the same test conditions as in the steps above, but without adding the compound to the medium (e.g., an equal volume of sterile water or saline is added instead). In such cases, a candidate compound that enhances DJ-1, Parkin or Pink-1 promoter activity can be identified as exhibiting a desired activity by exhibiting at least about 5% percent greater activity relative to the control; more preferably at least about 10% greater activity relative to the control assay; and still more preferably at least about 30%, at least about 80%, about 100%, about 150% or about 200% greater activity or more relative to the control.

Detection of the detectable reporter sequence in the in vitro assay can be achieved by one or a combination of different conventional methods. In one embodiment, the reporter produces an assay signal that is detected by a fluorometric assay. Typical of such fluorometric assays are those that include use of at least one of a fluorescence microscope, fluorometer, fluorescence microplate reader, fluorescence activated cell sorter or flow cytometer.

As will be apparent, it will often be helpful (but not always necessary) to include a suitable control experiment. In one such embodiment, the assay signal from the reporter sequence is compared to a baseline (control) signal produced by a control assay. In such instances, the baseline (control) signal is subtracted from the assay signal to produce a corrected signal indicative of presence (or absence if it is substantially less then the control) of the compound. It will be appreciated, however, that in instances in which the activity of a reporter sequence expressed by a particular recombinant cell or cell line of the invention is well known, use of a control may not be necessary.

### Screening Formats

The compositions and methods of the invention are readily adaptable for use in an automated, semi-automated, or manual screening format. In one embodiment, the method is conducted in a high throughput screening assay. Such an embodiment will often be preferred when the screen is intended to assay compound libraries. General methods for performing such high throughput screens (HTS) have been reported, for instance, by the Institute of Chemistry and Cell Biology (ICCB) of Harvard University. General disclosure relating to such screens has been reported, for instance, by J. C Yarrow, et al. (2003) in Combinatorial Chemistry, 6: 79 (disclosing particular chemical libraries, equipment, and screens for performing certain high throughput fluorescence detection strategies). See also Smith, R. A et al. (2004) Comb. Chem. High Throughput Screen. 7: 141; and U.S. Pat. Nos. 6,630,311 and 6,444,992 for additional disclosure relating to performing HTS analysis.

In one embodiment of a screen, preferably an HTS assay, the assay uses at least one of and preferably all of the following components (available from Sigma, St. Louis, Mo.; Perkin Elmer, for instance) (1) microtitre plates, (2) fluorometer, fluorescence microplate reader, flow cytometer and a luminometer. Suitable microtitre plates will include about 5000 recombinant cells or cells lines of the invention in about 50 microliters of medium. One or a combination of suitable candidate compounds are present in the medium at a concentration of between from about 0.1 to about 2000 micomolar, preferably about 10 to 50 micromolar, for instance.

In many HTS formats, the screening of a chemical library will be preferred. To handle data output efficiently, it will often be useful to interface with a computing device, preferably interfaced with the fluorometer, fluorescence microplate reader, flow cytometer or luminometer, typically to receive input from the assay and provide output data to a user. Such output data can be stored and optionally manipulated by the computer or outputted to the user in real-time.

Nearly any suitable screening assay of the invention can be scaled down to the appropriate format (96 or 384 well) for high throughput screening. One reason this may be suitable in some embodiments is that it has been found that the luciferase activity generated is directly correlated with the number of cells. Due to the use of tumor-derived cell lines as backbone cells in some assay embodiments, certain compounds, such as HDAC inhibitors, will reduce the cell viability at high doses and affect luciferase readout. To ameliorate such a deficiency, a CMV-driven β-galactosidase gene was expressed as an internal control for toxicity and cell numbers. Although some care must be taken to ensure that detected agents do not regulate the CMV promoter. The SH-SY5Y cell based assay system described below, for instance, is helpful for initial screening of compounds that can transcriptionally activate neuroprotective DJ-1 for PD. Besides HDAC inhibitors, other FDA approved medications, novel compounds, or herbal supplements with low side effects may activate DJ-1. The positive results from the screening can be confirmed by further analysis of DJ-1 protein levels, and the candidate compounds will be tested for their neuroprotective effects in cell culture or animal models.

It will often be helpful to combine the in vitro screens of the invention (including the HTS assays) with one or more in vivo testing strategies. Such testing typically includes administering a compound exhibiting acceptable activity in one or more in vitro assays to an accepted animal model of a human neurological disorder. See the following references for examples of such models: Sherer, T B et al. (2003) Neurosci. Lett. 341: 87 (rat rotenone model of PD); Sherer T. B. et al. (2002) J. Neurosci. 22: 7006 (rat model of PD with altered alpha-synuclein); Betarbet R. et al. (2000) Nat. Neurosci. 3: 1301. (pesticide model of PD); US Pat. Publication Nos. 2003217370 to Giasson et al. (Transgenic animal expressing alpha-synuclein and uses thereof); 2004093623 to Baekelandt, V. (Non-human animal disease models); and 2003056231 to Elizer, M et al. (Development of transgenic model for interventions in neurodegenerative diseases). Other animal models of Parkinson's disease have been disclosed. See Feany, M B et al. in Nature (2000) 404 (6776): 394; and Auluck, P K et al. in Science (2002) 295 (5556): 865 (disclosing a fruit fly models of PD); Lauwers, et al. (2003) Brain. Pathol. 13(3): 364 (reporting viral mediated rodent brain degeneration); Kirik, D. et al. (2003) PNAS (USA) 100(5) 2884 (also reporting viral mediated rodent brain degeneration); and Lakso, M et al. (2003) J. Neurosci. 86(1): 165. (reporting a C. elegans model of neuronal degeneration).

Typical mammalian models of neurological disorders suitable for use with the invention in vivo testing preferably show at least one of the following indicators: partial or complete degeneration of nigrostriatal pathway, raphe nuclei, locus ceruleus, and the motor nucleus of vagus; partial or complete degeneration of intrastriatal and cortical cholinergic neurons and GABA-ergic neurons. In these screening embodiments, preferred screening methods will further include selecting compounds that prevent, treat, or reduce the severity of at least one of these indications. Such compounds can be used therapeutically to treat, for instance, Parkinson's disease and Huntington's disease. Thus in embodiments in which testing one or more candidate compounds includes in vivo testing, the method will further include selecting compounds that reduce the severity of or delay the onset of the symptoms in the animal by at least about 10% compared to a control. Such methods can be used, for instance, to confirm activity of any of the candidate compounds disclosed herein such as HDAC, or derivative thereof.

The following Examples are intended to be illustrative of the scope of the present invention.

### EXAMPLE 1

### Expression Vector for Detecting Agents that Activate DJ-1

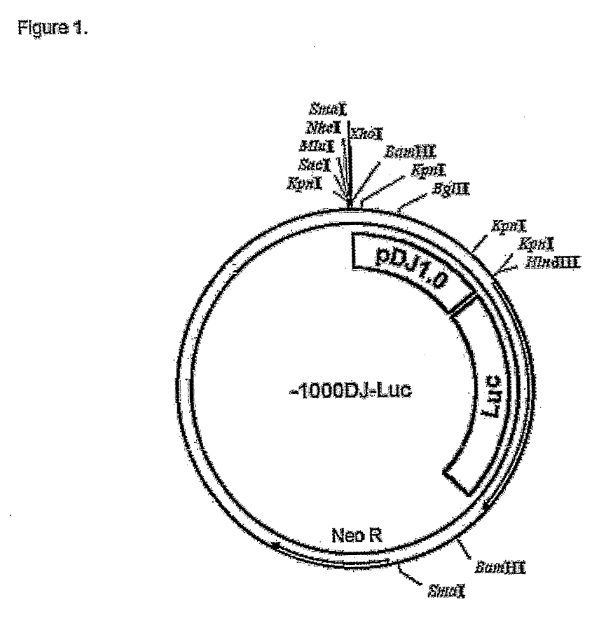

To develop a cell-based assay system where one can identify agents that transcriptionally activate DJ-1, a plasmid vector (−1000DJ-Luc) was made that expressed a luciferase reporter gene directed by the human DJ-1 promoter containing 1000 base pairs upstream and 65 base pairs downstream sequences (−1000 to +65) of the transcription initiation site (FIG. 1). A19 base pair sequence containing +46 to +65 of the DJ-1 gene was fused to the luciferase gene to generate the control plasmid (0DJLuc). A neomycin resistance gene was inserted into the vectors as a selection marker. Next, human dopaminergic SH-SY5Y neuroblastoma cells were transfected with either −1000DJLuc or 0DJLuc selected for stably transfected clones. A plasmid encoding β-galactosidase gene was co-transfected to serve as an internal control for luciferase expression.

### EXAMPLE 2

### Assay for Detecting Agents that Activate DJ-1

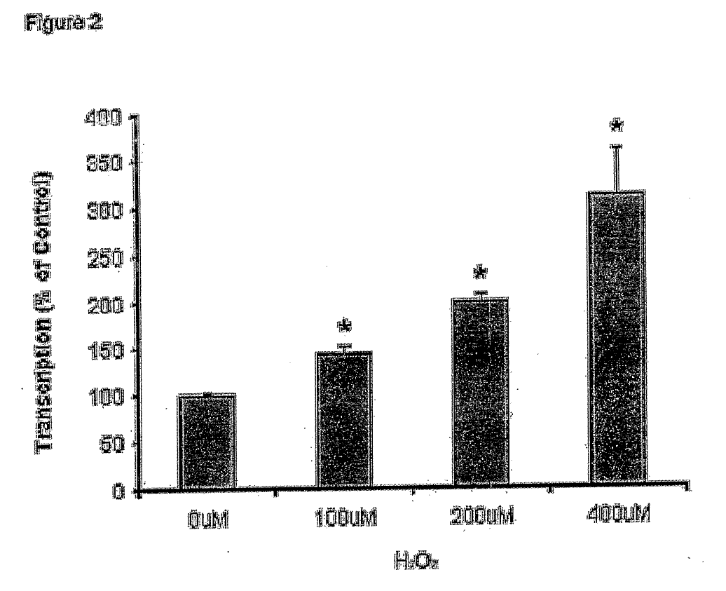

To confirm the stable expression of the reporter genes and to evaluate these cells as devices for drug discovery, stably transfected SH-SY5Y cells were treated with increasing doses of H₂O₂. This agent has been reported to upregulate DJ-1. See for example Reference 21. Twenty-four hours after treatment, cells were harvested to analyze luciferase and β-galactosidase activities. Toxicity induced by the higher dose H₂O₂ was corrected by reduced β-galactosidase activities. It was found that H₂O₂ activated the human DJ-1 promoter in a dose dependent manner (FIG. 2). SH-SY5Y cells stably expressing −1000DJ-Luc and a plasmid encoding CMV-β-galactosidase were treated with the indicated amount of H₂O₂ for 24 hours. Luciferase and β-galactosidase activities were then determined. Because cellular toxicity increases with increasing H₂O₂ dosages, luciferase activity was normalized to β-galactosidase activity, with the value of untreated sample designated as 100. The values shown represent the average of 2 experiments carried out in triplicate. (P<0.05 by Anova). The Luciferase activity in control cell lines expressing 0DJ-Luc was not affected by H₂O₂. To further validate the cells as tools for compound screening, a rational approach to selecting candidate compounds that can transactivate DJ-1 gene based on the analysis of the human DJ-1 promoter was taken.

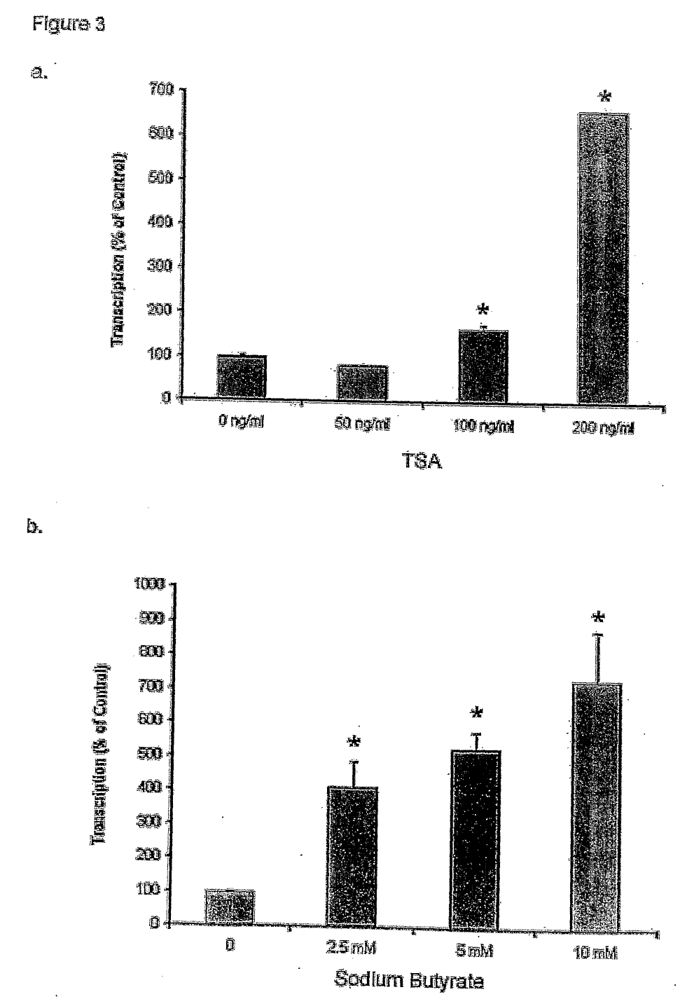

The −1000DJ-Luc-SH-SY5Y cells were treated with increasing concentrations of Trichostatin A (TSA), an organic zinc chelator that potently inhibits the zinc hydrolase activity of HDACs, or a structurally distinct HDAC inhibitor, sodium butyrate, and analyzed abation of the luciferases. SH-SY5Y cells stably expressing −1000DJ-Luc were treated with increasing doses of TSA (FIG. 3A) or sodium butyrate (FIG. 3B) for 24 hours. Results were analyzed as described in FIG. 2. DJ-1 promoter-driven luciferase activity was then assayed. Although the expression of internal control β-galactosidase gene, directed by the CMV promoter, was induced by HDAC inhibitors as reported, both TSA and sodium butyrate significantly activated the DJ-1 promoter (FIGS. 3A and 3B), even after the luciferase activity was normalized with β-galactosidase activity.

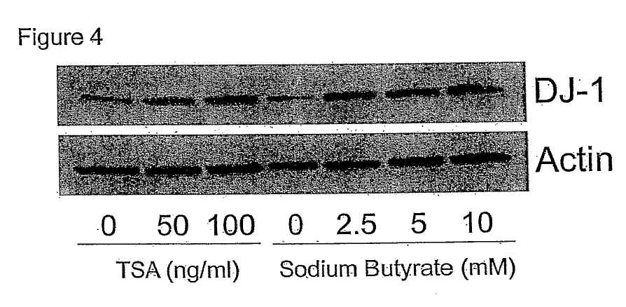

Consistent with the activation of the DJ-1 promoter, total endogenous DJ-1 protein levels increased markedly in these cells treated with TSA or sodium butyrate as shown in FIG. 4. FIG. 4 shows the accumulation of endogenous DJ-1 protein in cells treated with HDAC inhibitors. SHSY5Y cells stably expressing −1000DJ-Luc were treated with TSA or sodium butyrate for 24 hours. Total proteins were then extracted and analyzed by SDS-PAGE. After transfer, the membrane was first probed with a mouse monoclonal anti-DJ-1 antibody (1:1000) to reveal DJ-1 expression (Top panel). The membrane was then stripped and re-probed with a goat polyclonal anti-actin (1:1000). Therefore, and without wishing to be bound to theory, it is believed that by activating an anti-oxidative and neuroprotective protein DJ-1, HDAC inhibitors may prevent neuronal cell death in Parkinson disease or other neurodegenerative diseases.

See Van Lint, C. et al. (1996) Gene Expr. 5: 245; and Grassi, G et al. (2002) Biol. Pharm. Bull 25: 853.

FIG. 4 shows accumulation of endogenous DJ-1 protein in cells treated with HDAC inhibitors. SHSY5Y cells stably expressing −1000DJ-Luc were treated with TSA or sodium butyrate for 24 hours. Total proteins were extracted and analyzed by SDS-PAGE. After transfer, the membrane was first probed with a mouse monoclonal anti-DJ-1 antibody (1:1000) to reveal DJ-1 expression (Top panel). The membrane was then striped and re-probed with a goat polyclonal anti-actin (1:1000).

The following materials and methods were used as needed to perform Examples 1 and 2.

A. Plasmids and Chemicals. DJ-1 promoter sequences from −1000 to +65 relative to the transcriptional initiation site was amplified from pDJ-1(1)luc by PCR with Xho I and Hind III sites using the following primers: DJ-1 F, 5′ GGTGGTCTCGAGGGATCCTTCTAAGCTCATTCAAGA (SEQ ID NO:); DJ-1 R, 5′ GGAGGAAAGCTTTTGGGTACCACTCACCCCA (SEQ ID NO:). The PCR product was then inserted into pGL3basic vector (Promega), between Xho I and Hind III sites to generate 1000DJ-Luc vector. For 0DJ-Luc, the sequences from +46 to +65 relative to the transcriptional initiation site were inserted between XhoI and Hind III sites instead. To facilitate selection, a neomycin resistant gene directed by the SV40 promoter with BamHI and Xho I linker was inserted between the BamHI and SalI sites of pGL3 basic, with the original SalI site abolished after ligation. pON260 encoding β-galactosidase gene was described previously. H₂O₂, Trichostatin A (TSA), and sodium butyrate were obtained from Sigma.

Fluorometric assays of cell viability and cytotoxicity are easy to perform with the use of a fluorescence microscope, fluorometer, and fluorescence microplate reader or flow cytometer; and they offer many advantages over traditional calorimetric and radioactivity-based assays. Also discussed in this section are our unique single-step kits for assessing gram sign and for simultaneously determining gram sign and viability of bacteria.

For information relating to pDJ-1(1)Luc see Taira, T., et al. Gene 263, 285-92 (2001).

B. Cells and Transfection. The human neuroblastoma cell line SH-SY5Y was plated in 6 well dish at 70% confluency and co-transfected with 150 ng of pON260 and 25 fmol of −1000DJ-Luc or 0DJ-Luc per well with Transfectin reagent (Bio-Rad). 24 hours after transfection, cells were re-plated in 10 cm dishes and cultured in medium with 800 μg/ml of Geneticin (G418, Invitrogen). Three days later, cells were grown in medium containing 400 μg/ml of Geneticin. Surviving clones were cultured, and expanded, and subjected to analysis.

C. Luciferase and β-galactosidase Assays. Cells were treated with the indicated doses of H₂O₂, TSA or sodium butyrate for 24 hours before being lysed in reporter lysis buffer (Promega). Luciferase and β-galactosidase activities were determined using assay kits following manufacturer's protocol (Promega).

The following vectors have been disclosed as Genbank/Embl accession numbers. pGL3(R2.1)-Basic, AY487821; pGL3(R2.2)-Basic, AY487822; phRG(R2.1)-Basic, AY487823; phRG(R2.2)-Basic, AY487824.

The forgoing Examples and discussion shows, among other things, that it is now possible to develop a cell-based luciferase assay to screen for compounds that can activate a neuroprotective gene lost in some PD patients. As a proof of principle, it has been particularly shown that H₂O₂, which is known to upregulate DJ-1 expression, transactivates DJ-1 promoter. In addition, it is believed that HDAC inhibitors are good therapeutics for PD as evidenced by the assays. The corresponding increase in endogenous DJ-1 protein levels in cells treated with HDAC inhibitors further validated the assay as a reliable tool for drug discovery.

### EXAMPLE 3

### Assay for Detecting Agents that Activate Parkin

The foregoing disclosure (including Examples 1-3, above) is readily adapted to produce an in vitro assay that can be employed to detect compounds that activate the Parkin promoter or a functional portion thereof. In this embodiment, the following oligonucleotides are designed to amplify the Parkin promoter from a sample of genomic DNA.

Forward Primer:

(SEQ ID NO: )

5′-GGTGGTCTCGAGCGTAAATAATAAATTACGGAGTAAGGG-3′
Reverse Primer:

(SEQ ID NO: )

5′-GGAGGAAAGCTTAGGAACAGGCCCATGCGCGA-3′

More specifically, use of the forward and reverse primers will amplify the Parkin promoter and transcription initiation site downstream sequence from position −1488 to +68. Preferred amplification methods include PCR (polymerase chain reaction). See U.S. Pat. Nos. 4,683,195; and 4,683,202, for instance, for disclosure relating to performing PCR.

The amplified Parkin promoter and sequence downstream of the transcription start site can be inserted into the expression vectors disclosed herein using conventional recombinant technology. Specifically, between the XhoI and HindIII insertion sites. The resulting expression vector can be used to detect compounds that modulate the Parkin promoter.

### EXAMPLE 4

### Assay for Detecting Agents that Activate Pink-1

There are reports that mutations in the Pink-1 are associated with PD. See Valente, E M. Et al. (2004) Science 304(5674): 1158. Accordingly, making expression vectors that include the Pink-1 promoter (or functional portion thereof). The human genome data bank provides 5′ upstream flanking sequence for the human PINK-1 (PTEN induced putative kinase 1; Exon 1). The promoter can be readily cloned and used in accord with the invention by taking advantage of a unique identifier spanning about 5 kb upstream of the transcription initiations site at genomic location 20424423 to 20429423 base pair on human chromosome 1. The first ATG (transcription start site) is in exon 1 at 20429548 base pair of the chromosome.

The following oligonucleotide primers can be used to amplify the Pink-1 promoter sequence and transcription initiation site downstream sequence from position −1994 to +32.

Forward primer:

(SEQ ID NO: )

5′-GGTGGTCTCGAGCTGTCTCAGAGTGAGACAGTGGG-3′
Reverse primer:

(SEQ ID NO: )

5′-GGAGGAAAGCTTGGTCACAACAAACTTGGGGCGG-3′

The amplified Pink-1 promoter and sequence downstream of the transcription start site can be inserted into the expression vectors disclosed herein using conventional recombinant technology. Specifically, between the XhoI and HindIII insertion sites. The resulting expression vector can be used to detect compounds that modulate the Pink-1 promoter in one or more of the assays disclosed herein.

### EXAMPLE 5

### DJ-1 Protects Against H₂O₂ and α-Synuclein-Induced Neuronal Apoptosis

Human neuroblastoma SH-SY5Y cells were transfected with plasmids encoding GFP (Control) or His-tagged-DJ-1 (DJ-1), and treated with 150 μM of H₂O₂ for twenty-four hours or co-transfected with A30P α-synuclein. SH-SY5Y cells plated on coverslips in 24 well dishes were transfected using Lipofectamine 2000 (invitrogen) or Transfectin (Bio-Rad) reagents according to the manufacturer's instructions. To assess the protective effect of DJ-1 on α-synuclein toxicity, 2 μg of A30P α-synuclein was co-transfected with 1 μg of wild type Myc-His-tagged DJ-1 expression plasmids. For controls, equal amount of either GFP or pcDNA3 empty vector was used to match transfected DJ-1. Forty-eight hours after transfection, cells were fixed and double-labeled with anti α-synuclein (1:600, BD Bioscience) and anti-H is (1:600, Santa Cruz) antibodies, followed by immunofluorescence microscopy with appropriate Cy2/Cy3 conjugated secondary antibodies to identify cells overexpressing α-synuclein and DJ-1.

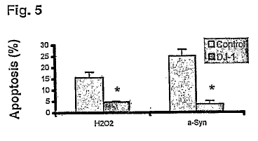

Apoptotic cells that were α-synuclein and DJ-1-positive were identified by characteristic nuclear morphology (chromatin condensation, nuclear fragmentation) after staining with the bisbenzimide DNA intercalator, Hoechst 33258 (1 μg/ml), as described in Xu et al, Nat Med, 2002. Apoptosis was scored by a blinded observer. Values shown in FIG. 5 represent the mean±S.E.M. n=3. For each experiment, at least 150 cells were scored for each condition. P<0.01 as indicated for WtDJ-1 relative to control. *P<0.05 for the indicated DJ-1 mutants relative to WtDJ-1 by ANOVA with post-hoc Student-Neumann-Kiels test.

To assess protection against oxidative stress, cultured SH-SY5Y cells were transfected with GFP (Control) or a Myc-His-tagged DJ-1, and treated with 250 μM of H₂O₂ for 24 hours before fixation and immunofluorescence microscopy and apoptosis analysis as described above. Values represent the mean±S.E.M. n=3.

As shown in FIG. 5, DJ-1 expression protected SH-SY5Y cells from apoptotic cell death in culture. Similar results were observed in human primary neuronal culture, which were enriched in dopaminergic neurons.

### EXAMPLE 6

### HDAC Inhibitors Activate the DJ-1 Promoter

SH-SY5Y cells stably expressing −1000DJ-Luc or 0DJ-Luc were treated with HDAC inhibitors suberoylanilide hydroxamic acid (SAHA), TSA and sodium butyrate for twenty-four hours. Cells were then lysed in passive lysis buffer (Promega), and the resulting lysates were used in luciferase and protein assays. The luciferase readings were normalized to total protein content.

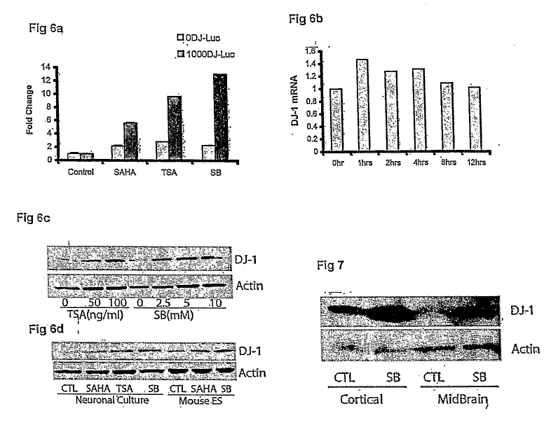

The HDAC inhibitors specifically activated luciferase expression directed by the human DJ-1 promoter in SH-SY5Y cells stably expressing the reporter construct (FIGS. 6A-6D). The raw data showed that the luciferase activity in 1000DJ-Luc cells treated with SAHA (5 μM), TSA (100 ng/ml) or sodium butyrate (SB) (5 mM) was >100 times higher than the luciferase activity present in the 0DJ-Luc control cells. The results shown in FIGS. 6A-6D were normalized with total protein content. The data represent the relative fold change relative to the values of untreated samples, which were normalized to 1.

### EXAMPLE 7

### HDAC Inhibitors Increase DJ-1 mRNA Expression

Cells treated with the HDAC inhibitor, sodium butyrate (5 mM), also exhibited a transient increase in DJ-1 mRNA levels. SH-SY5Y cells were treated with 5 mM of sodium butyrate. Cells were then harvested for mRNA one, two, four, eight, and twelve hours after treatment. DJ-1 mRNA levels were quantitated using real-time PCR. The results of these experiments are shown in FIG. 6B. DJ-1 mRNA levels were normalized to internal control β-actin mRNA levels.

### EXAMPLE 8

### HDAC Inhibitors Increase DJ-1 Protein Expression In Vitro

HDAC inhibitors also increased DJ-1 protein levels as shown in FIGS. 6C and 6D. SH-SY5Y cells (FIG. 6C), human primary neuronal cultures containing cortical neurons and glia (FIG. 6D), or mouse embryonic stem cells (FIG. 6D) were treated with increasing doses of TSA (0, 50, or 100 ng/ml) and sodium butyrate (0, 2.5, 5, or 10 mM). The cells were then lysed. The protein concentration of each sample was determined using a Protein DC assay (Bio-Rad). Equal amount of proteins (30-40 μg) were resolved by 4-20% Tris-glycine SDS-PAGE in each experiment. Proteins were transferred onto nitrocellulose membranes, and probed with antibodies against DJ-1 (Stressgen) or β-actin (Santa Cruz). DJ-1 protein was then visualized using chemiluminescent detection, ECL WESTERN BLOTTING DETECTION SYSTEM (Amersham, Piscataway, N.J.), according to the manufacturer's instructions. This treatment increased DJ-1 protein levels in a dose dependent fashion.

In accordance with the methods described herein, the invention provides for multiple assay formats of compound screening. In some embodiments, the DJ-1, Parkin, or Pink-1 promoter, or a fragment thereof, is endogenously expressed in the cell and expression is detected by assaying an mRNA level or assaying a protein level. In other embodiments, a heterologous DJ-1, Parkin, or Pink-1 promoter, or fragment thereof, is used. In various embodiments, the promoter is operably linked to a detectable reporter. Any standard method of assaying promoter activity may be used. For example, promoter expression may be detected by detecting the reporter.

### EXAMPLE 9

### HDAC Inhibitors Increase DJ-1 Protein Expression In Vivo

Sodium butyrate also increased DJ-1 protein levels in mouse brain in vivo as shown in FIG. 7. For analysis of DJ-1 protein levels in mouse brains, C57BL6 mice were injected intraperitoneally with vehicle (PBS) or sodium butyrate (1200 mg/kg) daily for fourteen days. Mice were euthanized and the cortical and midbrain tissues were collected, and snap frozen in liquid nitrogen. Tissues were disrupted in RIPA-DOC buffer with 1× protease inhibitor using a Dounce homogenizer. The protein concentration of each sample was determined using a Protein DC assay (Bio-Rad). Equal amounts of proteins (30-40 μg) were resolved by 4-20% Tris-glycine SDS-PAGE in each experiment. Proteins were transferred onto nitrocellulose membranes, and probed with antibodies against DJ-1 (Stressgen) or β-actin (Santa Cruz).

### EXAMPLE 10

### Use of the DJ-1 Luciferase Assay in a High Throughput Screen

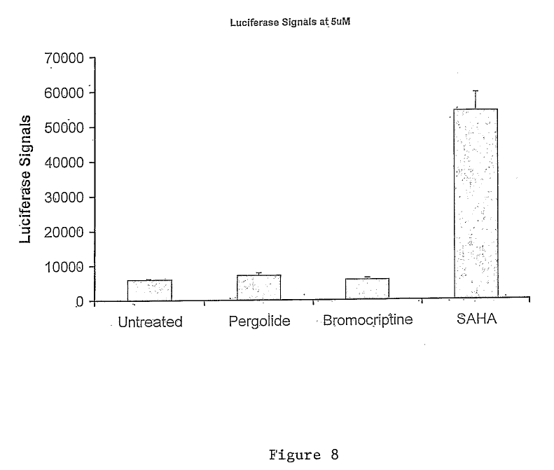

The assay described above was carried out in a 96-well format. Twelve replicate samples of 1000DJ-Luc cells were each treated with pergolide or bromocriptine, which served as negative controls, or with SAHA, a compound known to increase DJ-1 expression. 5 μM of each compound was added to the cells in a 30 μl volume. Cells were lysed in 100 μl/well of passive lysis buffer, and 20 μl aliquots of the cell lysates were then used for a luciferase assay. Bromocriptine and pergolide are dopamine receptor agonists used for the treatment of motor symptoms in Parkinson's disease. Unlike L-DOPA, these agonists are not pro-drugs, and consistent with this function, these compounds showed no neuroprotective activity in the DJ-1 luciferase assay (FIG. 8). Pergolide and bromocriptine treated cells produced little luciferase activity. In fact, levels of luciferase activity observed with these compounds were very similar to levels present in untreated control cells. In contrast, the SAHA treated cell samples produced substantial levels of luciferase activity. A Z′ value was calculated using bromocriptine as the negative control. The Z′ value is a statistical parameter that is derived from the differences between the means and standard deviations among signals and noises, and measures the ability of an assay to reproducibly distinguish active from inactive compounds. Z′ values greater than 0.5 indicate that the assay shows both specificity and robustness. The Z′ value of this assay was 0.790 indicating that the assay has excellent specificity and robustness, and should be useful for high throughput screening implementation. See “A Simple Statistical Parameter for Use in Evaluation and Validation of High Throughput Screening Assays” Zhang J H, Chung T D, Oldenburg K R, J Biomol Screen. 1999; 4(2):67-73.

### Transcriptional Assays

SH-SY5Y cells stably expressing −1000DJ-Luc or 0DJ-Luc were treated with the indicated HDAC inhibitors for 24 hours prior to harvest.

### Quantitative Real-Time PCR

Total RNA was extracted from the cells treated with sodium butyrate at various time points using Trizol reagent (Invitrogen). The total RNA was then purified with the RNAeasy kit (Qiagen). The quality of the RNA was confirmed using agarose (1%) gel electrophoresis. RNA (1 ng) from each sample was then amplified using Roche LightCycler and Qiagen QuantiTect™ SYBR Green RT-PCR kit according to the manufacturer's instructions. This protocol included: reverse transcription at 50° for 20 minutes, initial activation of Hotstar Taq polymerase at 95° C. for 15 minutes, then 40 cycles of 3-step cycling, including denaturation at 94° C. for 15 seconds; annealing at 58° C. for 20 seconds; extension at 72° C. for 20 sec). A fluorescence detection step was carried out at the end of each cycle. Melting curve analysis (0.1° C. per sec ramping rate, 60-95° C.) was performed with a continuous fluorescence measurement following the 40th cycle. β-actin mRNA was amplified in parallel as an internal control.

### HDAC Inhibitor Treatment and Analysis of DJ-1 Protein Levels

Human SH-SY5Y cells, a primary neuronal culture containing dopaminergic neurons and glia, or mouse embryonic stem cells were treated with selected HDAC inhibitors for 24 hours. Cells were then lysed in RIPA-DOC buffer (50 mM Tris, pH 7.2; 150 mM NaCl; 1% Triton-X100, 1% deoxycholate and 0.1% SDS) with 1× fresh protease inhibitor cocktail. For analysis of DJ-1 protein levels in mouse brains, C57BL6 mice were injected intraperitoneally with sodium butyrate (1200 mg/kg) or vehicle (PBS) daily for 14 days. Mice were euthanized and the cortical and midbrain tissues were collected, and snap frozen in liquid nitrogen. Tissues were disrupted in RIPA-DOC buffer with 1× protease inhibitor using Dounce homogenizers. Protein concentration of each sample was determined using Protein DC assay (Bio-Rad). Equal amounts of proteins (30-40 μg) were resolved by 4-20% Tris-glycine SDS-PAGE in each experiment. Proteins were transferred onto nitrocellulose membranes, probed with antibodies against DJ-1 (Stressgen) or β-actin (Santa Cruz) and visualized using chemiluminescence (ECL WESTERN BLOTTING DETECTION SYSTEM, Amersham, Piscataway, N.J.) in accordance with the manufacturer's instructions.

A review of the following specific references will help advance appreciation of the present invention.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of identifying a compound that increases DJ-1 gene expression in a cell, the method comprising:
(a) contacting a cell expressing a DJ-1 promoter with a candidate compound; and
(b) detecting an increase in DJ-1 gene expression, wherein an increase in the level of DJ-1 gene expression in the cell relative to a reference, identifies the candidate compound as increasing DJ-1 expression.

2. The method claim 1, wherein the DJ-1 promoter is a heterologous promoter operably linked to a detectable reporter present in an expression vector.

3-5. (canceled)

6. The method of claim 1, wherein the DJ-1 promoter is endogenously expressed in the cell.

7-8. (canceled)

9. A method of identifying a compound that increases Parkin gene expression in a cell, the method comprising:
(a) contacting a cell expressing a Parkin promoter with a candidate compound; and
(b) detecting Parkin gene expression, wherein an increase in the level of Parkin gene expression in the cell relative to a reference, identifies the candidate compound as a compound that increases Parkin gene expression.

10. (canceled)

11. The method of claim 10, wherein the Parkin promoter is present in an expression vector operably linked to a detectable reporter.

12-14. (canceled)

15. The method claim 14, wherein Parkin gene expression is detected by assaying mRNA level or protein level.

16. (canceled)

17. A method of identifying a compound that increases Pink-1 gene expression in a cell, the method comprising:
(a) contacting a cell expressing a Pink-1 promoter with a candidate compound; and
(b) detecting Pink-1 gene expression, wherein an increase in the level of Pink-1 gene expression in the cell relative to a reference, identifies the candidate compound as a compound that increases Pink-1 gene expression.

18. The method claim 17, wherein the Pink-1 promoter is a heterologous promoter operably linked to a detectable reporter present in an expression vector.

19-25. (canceled)

26. A method for identifying a compound that treats or prevents a neurological disorder in a subject, the method comprising:
(a) contacting a cell comprising a DJ-1, Parkin, or Pink-1 promoter operably linked to a detectable reporter with a candidate compound; and
(b) detecting a change in the expression of the reporter sequence relative to a control, thereby identifying a compound that treats or prevents a neurological disorder.

27-35. (canceled)

36. The method of claim 26, wherein the candidate compound is a histone deacetylase inhibitor (HDAC).

37. The method of claim 26, wherein the candidate compound is a short-chain fatty acid, hydroxamic acid, cyclic tetrapeptide, or benzamide.

38. (canceled)

39. The method of claim 26, wherein the candidate compound is 4-phenylbutyrate, valproic acid, suberoylanilide hydroxamic acid (SAHA), pyroxamide, trochostatin A, oxamflatin, trapoxin A, apicidin, butyrate salt; or a derivative thereof.

40. The method of claim 26, wherein the method further comprises testing the compound in an animal model.

41. The method of claim 40, wherein the compound is administered to an animal before, during or after exposure to an amount of 1-methyl-4-phenyl-1,2,3,6-tetrahydropyridine (MPTP) sufficient to cause symptoms associated with Parkinson's disease in the animal.

42. The method of claim 40, further comprising administering the compound to an animal before, during or after exposure to an amount of rotenone sufficient to cause symptoms associated with Parkinson's disease in the animal.

43. The method of claim 40, wherein further testing comprises administering the compound to a transgenic animal expressing α-synuclein.

44. The method of claim 40, wherein the animal is a rodent.

45. The method of claim 44, wherein Parkinson's disease is assayed by detecting degeneration of a nigrostriatal pathway, raphe nuclei, locus ceruleus, or motor nucleus of vagus.

46. The method of claim 20, wherein the method further comprises selecting compounds that treat or prevent at least one symptom of Parkinson's disease.

47. (canceled)

48. The method of claim 40, wherein the method further comprises selecting a compound that reduces the severity of or delays the onset of a Parkinson's disease symptom in the animal by at least about 10% compared to a control.

49. The method claim 48, wherein the method is used to confirm that an HDAC inhibitor can prevent or treat Parkinson's Disease (PD).

50. An expression vector comprising at least one of a DJ-1, Parkin or Pink-1 promoter sequence operably linked to at least one reporter sequence.

51. The expression vector of claim 50, wherein the DJ-1, Parkin or Pink-1 promoter sequence comprises about 2000 base pairs upstream of the DJ-1 Parkin or Pink-1 transcription start site.

52. The expression vector of claim 50, wherein the DJ-1, Parkin or Pink-1 promoter sequence comprises about 1500 base pairs upstream of the DJ-1, Parkin, or Pink-1 transcription start site.

53-69. (canceled)

70. The expression vector of claim 50, wherein the DJ-1, Pink1, or Parkin promoter is operably linked in sequence to:
1) a polynucleotide encoding an ampicillin resistance gene or functional fragment thereof;
2) an f1 origin sequence;
3) an upstream synthetic poly(A) region;
4) a promoter,
5) a polynucleotide sequence encoding a luciferase derivative;
6) an SV40 late poly (A) signal; and
7) a polynucleotide encoding a neomycin resistance gene; or functional fragment thereof.

71-81. (canceled)
